(19) United States
(12) Patent Application Publication
(10) Pub. No.: US 2019/0282558 A1
(43) Pub. Date: Sep. 19, 2019

(54) Methods of Treating Zika Virus Infection

(71) Applicant: Emergent Virology LLC, Gaithersburg, MD (US)

(72) Inventors: Anthony Michael TRESTON, Rockville, MD (US); Kelly Lyn WARFIELD, Adamstown, MD (US)

(73) Assignee: Emergent Virology LLC, Gaithersburg, MD (US)

(21) Appl. No.: 16/301,924

(22) PCT Filed: May 16, 2017
(86) PCT No.: PCT/US2017/032909

§ 371 Date: Nov. 15, 2018

Related U.S. Application Data
(60) Provisional application No. 62/337,173, filed on May 16, 2016.

(51) Int. Cl. A61K 31/4525; A61P 31/14; A61K 31/445; A61K 31/454; A61K 31/4545; A61K 31/5377; A61K 31/52

(52) U.S. Cl. CPC A61K 31/4525; A61P 31/14; A61K 31/445; A61K 31/52; A61K 31/4545; A61K 31/5377; A61K 31/454

(57) ABSTRACT

This disclosure provides methods for treating or preventing a disease or condition caused by or associated with a Zika virus infection in a subject. The methods can comprise administering to a subject in need thereof an effective amount of an iminosugar compound as described herein.

### FIELD

The present disclosure is directed to methods of treating Zika virus infections comprising administering to a subject in need thereof an effective amount of an iminosugar compound as described herein.

### BACKGROUND

Zika virus (Zika) is a mosquito-borne flavivirus first isolated in a nonhuman primate in 1947 and in mosquitos in 1948. Fewer than 20 human cases were documented in Africa and Asia for the first 50 years following its discovery. In 2007, an outbreak of Zika virus-associated fever occurred on the western Pacific island of Yap, which was followed by a larger outbreak in French Polynesia in 2014-15. Zika virus emerged in the Americas in the last 2 years with more than 50 countries reporting autochthonous transmission.

Infection with Zika virus has been associated with severe neurologic complications. For example, an increase in Guillain-Barré syndrome was observed in adults in French Polynesia. More recently, the CDC concluded that Zika virus infection in pregnant women is responsible for microcephaly in gestating fetuses.

Zika only recently emerged as a threat in the western hemisphere, there are currently no specific treatments or preventative vaccines for Zika virus infections. Thus, new therapeutic agents for treating Zika are urgently needed.

### BRIEF SUMMARY OF THE INVENTION

The present disclosure is directed to methods of treating Zika virus infection comprising administering to a patient in need thereof a compound according to Formula I:

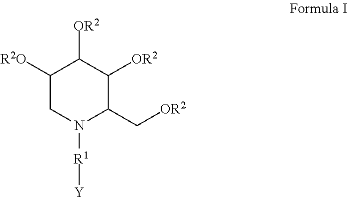

wherein R¹, R², and Y are defined herein.

In certain embodiments, the present disclosure provides a method of treating a Zika virus infection, the method comprising administering to a subject in need thereof, an effective amount of a compound according to Formula I:

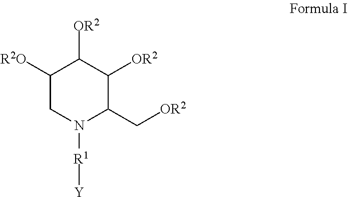

wherein:
R¹ is C₁-C₁₀ alkyl;
each R² is independently H, C₁-C₆ alkyl, or C₁-C₆ perfluoroalkyl;
Y is NR³R⁴, optionally substituted heterocyclyl, or C(R³)₂R⁵;
each R³ is independently H or C₁-C₆ alkyl;
R⁴ is

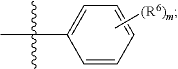

m is 0, 1, 2, 3, 4 or 5;
R⁵ is —O(C₁-C₄ alkyl);
R⁶ is, independently at each occurrence, NO₂, N₃, optionally substituted heteroaryl, optionally substituted heterocyclyl,

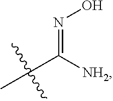 [SMILES: */C(N)=N/O]

—(CH₂)ₚN(R⁷)₂,

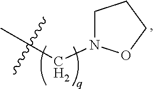

### I, Br, Cl, or F;

p is 1, 2, or 3;
q is 1, 2, or 3; and
R⁷ is, independently at each occurrence, H or C₁-C₆ alkyl, or, when taken together along with the nitrogen to which they are bound, is the group

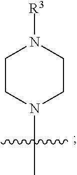

or a pharmaceutically acceptable salt thereof.

In certain embodiments, the compound of Formula I is a compound of Formula II:

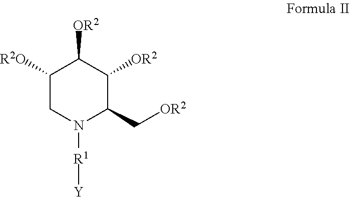

In certain embodiments, R¹ is selected from the group consisting of —CH₂, —CH₂CH₂—, —(CH₂)₃—, —(CH₂)₄—, —(CH₂)₅—, —(CH₂)₆—, —(CH₂)₇—, —(CH₂)₈—, —(CH₂)₉—, and —(CH₂)₁₀—.

In certain embodiments, R¹ is —(CH₂)₄—, —(CH₂)₆—, or —(CH₂)₈—.

In certain embodiments, R¹ is —(CH₂)₈— and Y is C(R³)₂R⁵.

In certain embodiments, each R³ is H and R⁵ is —O(C₁-C₄ alkyl), wherein the C₁-C₄ alkyl is selected from the group consisting of methyl, ethyl, n-propyl, isopropyl, butyl, sec-butyl, and t-butyl.

In certain embodiments, R¹ is —(CH₂)₆— and Y is NR³R⁴.

In certain embodiments, R³ is H and R⁴ is

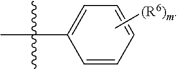

In certain embodiments, m is 1, 2, or 3, and R⁶ is independently at each occurrence NO₂, N₃, optionally substituted heteroaryl, optionally substituted heterocyclyl,

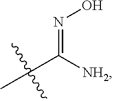 [SMILES: */C(N)=N/O]

—(CH₂)ₚN(R⁷)₂,

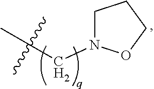

### I, Br, Cl, or F.

In certain embodiments, m is 3 and each R⁶ is halogen. In still further embodiments, the halogen is fluorine.

In certain embodiments, m is 2, at least one R⁶ is NO₂, and a second R⁶ is N₃, 2H-1,2,3-triazol-2-yl,

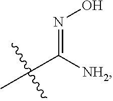 [SMILES: */C(N)=N/O]

1H-tetrazol-1-yl, 1H-pyrazol-1-yl, optionally substituted heterocyclyl, —(CH₂)ₚN(R⁷)₂, or

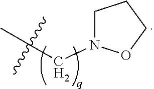

In certain embodiments, m is 1 and R⁶ is optionally substituted heteroaryl.

In certain embodiments, the optionally substituted heteroaryl is pyrimidin-2-yl or 2H-tetrazol-2-yl.

In certain embodiments, R¹ is —(CH₂)₈— and Y is optionally substituted heterocyclyl or C(R³)₂R⁵.

In certain embodiments, Y is tetrahydrofuran-2-yl.

In certain embodiments, Y is C(R³)₂R⁵.

In certain embodiments, R³ is independently at each occurrence, H or C₁-C₆ alkyl.

In certain embodiments, R³ is independently at each occurrence, methyl, ethyl, n-propyl, isopropyl, n-butyl, sec-butyl, or t-butyl.

In certain embodiments, R³ is methyl at each occurrence.

In certain embodiments, R⁵ is methoxy.

In certain embodiments, R¹ is —(CH₂)₄—.

In certain embodiments, the compound is selected from the group consisting of (2R,3R,4R,5S)-2-(hydroxymethyl)-1-(9-methoxynonyl)piperidine-3,4,5-triol; (2R,3R,4R,5S)-1-(6-((4-azido-2-nitrophenyl)amino)hexyl)-2-(hydroxymethyl)piperidine-3,4,5-triol; (2R,3R,4R,5S)-2-(hydroxymethyl)-1-(8-(tetrahydrofuran-2-yl)octyl)piperidine-3,4,5-triol; (2R,3R,4R,5S)-2-(hydroxymethyl)-1-(9-methoxy-9-methyldecyl)piperidine-3,4,5-triol; (2R,3R,4R,5S)-2-(hydroxymethyl)-1-(6-((2-nitro-4-(2H-1,2,3-triazol-2-yl)phenyl)amino)hexyl)piperidine-3,4,5-triol; (E)-N′-hydroxy-3-nitro-4-((6-((2R,3R,4R,5S)-3,4,5-trihydroxy-2-(hydroxymethyl)piperidin-1-yl)hexyl)amino)benzimidamide; (2R,3R,4R,5S)-2-(hydroxymethyl)-1-(6-((2-nitro-4-(1H-tetrazol-1-yl)phenyl)amino)hexyl)piperidine-3,4,5-triol; (2R,3R,4R,5S)-2-(hydroxymethyl)-1-(6-((2-nitro-4-(2H-tetrazol-2-yl)phenyl)amino)hexyl)piperidine-3,4,5-triol; (2R,3R,4R,5S)-2-(hydroxymethyl)-1-(6-((2-nitro-4-(pyrimidin-2-yl)phenyl)amino)hexyl)piperidine-3,4,5-triol; (2R,3R,4R,5S)-2-(hydroxymethyl)-1-(6-((4-morpholino-2-nitrophenyl)amino)hexyl)piperidine-3,4,5-triol; (2R,3R,4R,5S)-2-(hydroxymethyl)-1-(6-((2-nitro-5-(1H-tetrazol-1-yl)phenyl)amino)hexyl)piperidine-3,4,5-triol; (2R,3R,4R,5S)-2-(hydroxymethyl)-1-(6-((2-nitro-5-(piperazin-1-yl)phenyl)amino)hexyl)piperidine-3,4,5-triol; (2R,3R,4R,5S)-1-(6-((4-((dimethylamino)methyl)-2-nitrophenyl)amino)hexyl)-2-(hydroxymethyl)piperidine-3,4,5-triol; (2R,3R,4R,5S)-1-(6-((5-azido-2-nitrophenyl)amino)hexyl)-2-(hydroxymethyl)piperidine-3,4,5-triol; (2R,3R,4R,5S)-2-(hydroxymethyl)-1-(6-((3-nitro-4-(1H-pyrazol-1-yl)phenyl)amino)hexyl)piperidine-3,4,5-triol; (2R,3R,4R,5S)-2-(hydroxymethyl)-1-(6-((3-nitro-4-(1H-tetrazol-1-yl)phenyl)amino)hexyl)piperidine-3,4,5-triol; (2R,3R,4R,5S)-2-(hydroxymethyl)-1-(6-((4-((4-methylpiperazin-1-yl)methyl)-2-nitrophenyl)amino)hexyl)piperidine-3,4,5-triol; (2R,3R,4R,5S)-1-(6-((5-((dimethylamino)methyl)-2-nitrophenyl)amino)hexyl)-2-(hydroxymethyl)piperidine-3,4,5-triol; (2R,3R,4R,5S)-1-(4-((4-azido-2-nitrophenyl)amino)butyl)-2-(hydroxymethyl)piperidine-3,4,5-triol; (2R,3R,4R,5S)-2-(hydroxymethyl)-1-(6-((2,4,6-trifluorophenyl)amino)hexyl)piperidine-3,4,5-triol; (2R,3R,4R,5S)-1-butyl-2-(hydroxymethyl)piperidine-3,4,5-triol; (2R,3R,4R,5S)-2-(hydroxymethyl)-1-nonylpiperidine-3,4,5-triol; (2R,3R,4R,5S)-2-(hydroxymethyl)-1-(6-propoxyhexyl)piperidine-3,4,5-triol; (2R,3R,4R,5S)-1-(6-((4-(2H-tetrazol-2-yl)phenyl)amino)hexyl)-2-(hydroxymethyl)piperidine-3,4,5-triol; (2R,3R,4R,5S)-1-(6-((7H-purin-2-yl)amino)hexyl)-2-(hydroxymethyl)piperidine-3,4,5-triol; (2R,3R,4R,5S)-2-(hydroxymethyl)-1-(6-((4-(isoxazolidin-2-ylmethyl)-2-nitrophenyl)amino)hexyl)piperidine-3,4,5-triol; (2R,3R,4R,5S)-2-(hydroxymethyl)-1-(6-((2-nitro-4-(pyridin-2-yl)phenyl)amino)hexyl)piperidine-3,4,5-triol; (2R,3R,4R,5S)-2-(hydroxymethyl)-1-(6-((2-nitro-4-(pyridazin-3-yl)phenyl)amino)hexyl)piperidine-3,4,5-triol; (2R,3R,4R,5S)-2-(hydroxymethyl)-1-(6-((2-nitro-4-(pyrimidin-4-yl)phenyl)amino)hexyl)piperidine-3,4,5-triol; (2R,3R,4R,5S)-2-(hydroxymethyl)-1-(6-((2-nitro-4-(pyridin-3-yl)phenyl)amino)hexyl)piperidine-3,4,5-triol; and pharmaceutically acceptable salts thereof.

In certain embodiments, the subject is a human.

In certain embodiments, the human is a human female.

The present disclosure is also directed to methods of inhibiting Zika virus comprising administering to a subject in need thereof an effective amount of a compound according to Formula I or Formula II, or a pharmaceutically acceptable salt thereof. In certain embodiments, the subject is a human. In certain embodiments, the human is a human female.

The present disclosure is further directed to methods of inhibiting replication of Zika virus comprising administering to a subject in need thereof an effective amount of a compound according to Formula I or Formula II, or a pharmaceutically acceptable salt thereof. In certain embodiments, the subject is a human. In certain embodiments, the human is a human female.

### BRIEF DESCRIPTION OF THE DRAWINGS

The foregoing summary, as well as the following detailed description of the embodiments, will be better understood when read in conjunction with the appended figures. For the purpose of illustration, the figures may describe the use of specific embodiments. It should be understood, however, that the methods described herein are not limited to the precise embodiments discussed or described in the figures.

FIG. 1 is a graph showing the efficacy of UV-4 against Zika virus infection in mice.

FIGS. 2A and 2B show the in vitro inhibitory effect of UV-4 and UV-5 against Zika virus, respectively.

FIGS. 3A and 3B show the in vitro inhibitory effect of compounds 1 and 2 against Zika virus, respectively.

FIGS. 4A and 4B show the in vitro inhibitory effect of compounds 3 and 4 against Zika virus, respectively.

FIGS. 5A and 5B show the in vitro inhibitory effect of compounds 5 and 6 against Zika virus, respectively.

FIGS. 6A and 6B show the in vitro inhibitory effect of compounds 7 and 8 against Zika virus, respectively.

FIGS. 7A and 7B show the in vitro inhibitory effect of compounds 9 and 10 against Zika virus.

FIGS. 8A and 8B show the in vitro inhibitory effect of compounds 11 and 12 against Zika virus, respectively.

FIGS. 9A and 9B show the in vitro inhibitory effect of compounds 13 and 14 against Zika virus, respectively.

FIGS. 10A and 10B show the in vitro inhibitory effect of compounds 15 and 16 against Zika virus, respectively.

FIGS. 11A and 11B show the in vitro inhibitory effect of compounds 17 and 18 against Zika virus, respectively.

FIGS. 12A and 12B show the in vitro inhibitory effect of compounds 19 and 20 against Zika virus. respectively.

FIGS. 13A and 13B show the in vitro inhibitory effect of compounds 21 and 22 against Zika virus, respectively.

FIGS. 14A and 14B show the in vitro inhibitory effect of compounds 23 and 24 against Zika virus, respectively.

FIG. 15 shows the in vitro inhibitory effect of compound 25 against Zika virus.

### DETAILED DESCRIPTION

### Definitions

As used herein, “a” or “an” means one or more unless otherwise specified.

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

As used herein, the phrase “viral infection” describes a diseased state in which a virus such as Zika virus invades a cell and uses the cell's machinery to multiply or replicate, ultimately resulting in the release of new viral particles. This release results in the infection of other cells by the newly produced particles. Latent infection by certain viruses is also a possible result of viral infection.

As used herein, the terms “treat,” “treating,” and “treatment” refer to administering a therapy in an amount, manner, or mode effective to improve a condition, symptom, or parameter associated with a disease or disorder. Thus, “treating” a disease or condition caused by or associated with Zika virus means inhibiting the replication of the virus, inhibiting viral transmission, and/or ameliorating, alleviating, or otherwise improving the symptoms of a disease or condition caused by or associated with the virus. In some embodiments, the treatment can be considered therapeutic if there is a reduction in viral load, and/or a decrease in mortality and/or morbidity.

The term “therapeutically effective amount” as used herein refers to an amount of a compound disclosed herein that is sufficient to efficaciously treat Zika virus infection in an individual in need thereof.

As used herein, the term “preventing” refers to precluding a patient from getting a disorder and/or related symptoms, causing a patient to remain free of a disorder and/or related symptoms for a longer period of time, or halting the progression of a disorder, to either a statistically significant degree or to a degree detectable to one skilled in the art.

The term “subject(s)” as used herein refers to any animal that is susceptible to infection with Zika virus, such as a mammal, and more particularly, a human. Unless otherwise specified, the dosing amounts described herein are for adult subjects, e.g., adult humans. As understood by those skilled in the art, the dosing amounts described herein may be adjusted for a pediatric patient. In certain embodiments, the subject can be a pregnant mammal, and in particular embodiments, a pregnant human female.

As used herein, the term “UV-4” refers to the chemical compound having the structure:

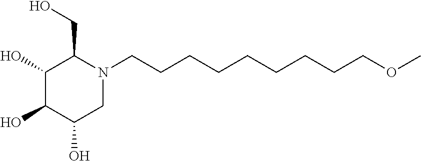 [SMILES: COCCCCCCCCCN1C[C@H](O)[C@@H](O)[C@H](O)[C@H]1CO]

Chemically, UV-4 is known as (2R,3R,4R,5S)-2-(hydroxymethyl)-1-(9-methoxynonyl)-piperidine-3,4,5-triol or N-(9-methoxynonyl)-deoxynojirimycin or N-(9-methoxynonyl)-1,5-dideoxy-1,5-imino-D-glucitol. UV-4 has been previously described, for example, in U.S. Application Publication No. 2011/0065752, as useful for treating Influenza virus infections. It was likewise described as useful for treating dengue viral infections in U.S. Pat. No. 8,450,345. UV-4 is a basic molecule and acid addition salts of UV-4 can be prepared, for example, using any known pharmaceutically acceptable acid.

The phrase “UV-4B” refers to the hydrochloride salt of UV-4.

As used herein, “aryl” or “aromatic,” groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups may be substituted or unsubstituted.

As used herein, “alkyl” means straight chain and branched saturated chains having from 1 to about 20 carbon atoms, and typically from 1 to 12 carbons or, in some embodiments, from 1 to 8 carbon atoms or from 1 to 4 carbons, designated Cₓ-C_{y} alkyl. Alkyl groups may be substituted or unsubstituted, as specified herein. Examples of straight chain alkyl groups include, but are not limited to, methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups.

As used herein the term “haloalkyl” is an alkyl group having one or more halo groups.

As used herein, the term “per-haloalkyl” refers to an alkyl group wherein all hydrogen atoms have been replaced with a halogen. For example, per-fluoro alkyl refers to an alkyl group in which all hydrogen atoms on the alkyl have been replaced with fluorine. Examples of per-fluoro alkyls include, but are not limited to, —CF₃ (trifluoromethyl), —CF₂CF₃ (pentafluoroethyl), etc.

As used herein, “phenyl” refers to a 6-membered cyclic aromatic hydrocarbon that does not contain heteroatoms. Phenyl rings can be substituted or unsubstituted. When substituted, the phenyl group can be mono, di, tri, tetra, or penta substituted and these substituents can be the same or can be different. When the phenyl ring is disubstituted, either with the same or different substituents, the phenyl ring can be 2,3-substituted, 2,4-substituted, 2,5-substituted, 2,6-substituted, 3,4-substituted, 3,5-substituted, or 3,6-substituted. When tri-substituted, whether with the same or different substituents, the phenyl ring can be 2, 3, 4-substituted, 3, 4, 5, substituted, 2,4,6-substituted, or 2,4,5-substituted.

As used herein, “heterocyclyl” refers to non-aromatic ring compounds containing 3 or more atoms, at least one of which is a heteroatom such as, but not limited to, N, O, or S. In some embodiments, the heterocyclyl group can contain 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass partially unsaturated and saturated ring systems, such as, for example, imidazolinyl and imidazolidinyl groups. The phrase also includes bridged polycyclic ring systems containing a heteroatom such as, but not limited to, quinuclidyl. As specified herein, a given heterocyclyl group can be optionally substituted. Exemplary heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, pyrrolinyl, piperidyl, piperazinyl, morpholinyl, thiomorpholinyl, tetrahydropyranyl, and tetrahydrothiopyranyl groups. The point of attachment of a given heterocyclyl group can be any appropriate atom and each such embodiment for a given heterocyclyl group is specifically contemplated. For example, tetrahydrofuranyl includes both tetrahydrofuran-2-yl as well as tetrahydrofuran-3-yl. The heteroatom(s) in a given heterocyclyl group can be optionally oxidized, if chemically possible.

As used herein, “heteroaryl” refers to aromatic ring compounds containing 5 or more atoms, at least one of which is a heteroatom such as, but not limited to, N, O, or S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, imidazolyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. The term “heteroaryl” also includes fused ring compounds in which all rings are aromatic such as indolyl groups and fused ring compounds in which only one of the rings is aromatic, such as 2,3-dihydro indolyl groups. The point of attachment of a given heteroaryl group can be any appropriate atom. Exemplary heteroaryl groups showing various points of attachment suitable for use in the present compounds are shown below:

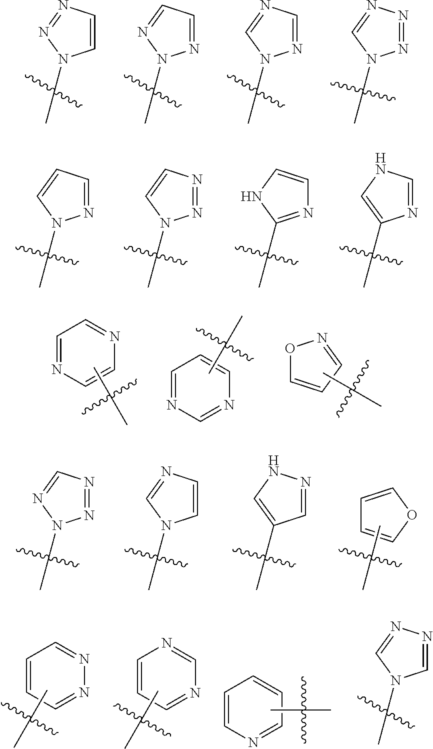

As used herein, the term “halogen” or “halo” refers to bromine, chlorine, fluorine, or iodine. In some embodiments, the halogen is fluorine. In other embodiments, the halogen is chlorine or bromine. The term “halide” as used herein refers to the anion of a halogen, such as bromide, chloride, fluoride, and iodide. In some embodiments, the halide is chloride or iodide.

As used herein, the term “optionally substituted” means that an identified group optionally contains one or more non-hydrogen substituents substituted for hydrogen. Examples of non-hydrogen substituent groups include: halogens (i.e., F, Cl, Br, and I); per-haloalkyl groups, hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, the phrase “pharmaceutically acceptable salt” refers to a salt of compound described herein with a pharmaceutically acceptable acid. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts that can be formed with the compounds disclosed herein include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides.

As used herein, “IC₅₀” refers to the concentration of a therapeutic agent, such as an iminosugar, used to achieve 50% reduction of viral load.

As used herein, “CC₅₀” (cytotoxic concentration 50) is a concentration of a therapeutic agent, such as an iminosugar, which results in the death of 50% of cells.

Iminosugars are well described in the art as being useful for treating various viral infections. The compound UV-4, for example, has been widely described as being useful for treating influenza virus infections and dengue virus infections. Despite the utility of various iminosugars for treating viral infections, it is extremely difficult to predict which iminosugars will be effective for treating a given viral disease.

It has now, however, been surprisingly discovered that the iminosugars described herein are suitable for treating Zika virus infections in a subject, and in particular, in humans.

Thus, the present disclosure provides methods of treating Zika virus infection in a subject in need thereof comprising administering an effective amount of a compound disclosed herein.

In particular embodiments, the present disclosure provides a method of treating Zika virus comprising administering to a subject in need thereof an effective amount of a compound of Formula I. Compounds of Formula I have the structure:

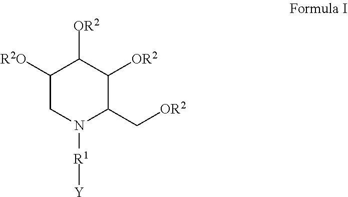

wherein

R¹ is C₁-C₁₀ alkyl;

each R² is independently H, C₁-C₆ alkyl, or C₁-C₆ perfluoroalkyl;

Y is NR³R⁴, optionally substituted heterocyclyl, or C(R³)₂R⁵;

each R³ is independently at each occurrence H or C₁-C₆ alkyl;

R⁴ is

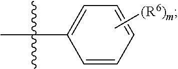

m is 0, 1, 2, 3, 4 or 5;

R⁵ is —O(C₁-C₄ alkyl);

R⁶ is, independently at each occurrence, NO₂, N₃, optionally substituted heteroaryl, optionally substituted heterocyclyl

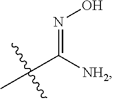 [SMILES: */C(N)=N/O]

—(CH₂)ₚN(R⁷)₂,

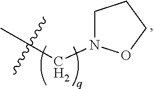

### I, Br, Cl, or F;

p is 1, 2, or 3;

q is 1, 2, or 3; and

R⁷ is, independently at each occurrence, H or C₁-C₆ alkyl, or, when taken together along with the nitrogen to which they are bound, is the group

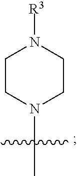

or a pharmaceutically acceptable salt thereof.

In particular embodiments, the compound of Formula I can be a compound of Formula II:

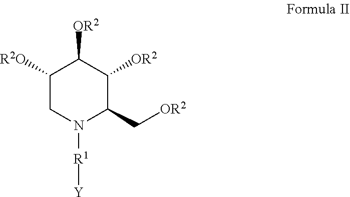

In certain embodiments, R¹ in the compound of Formulas I or II can be a C₁-C₁₀ alkyl selected from the group consisting of —CH₂—, —CH₂CH₂—, —(CH₂)₃—, —(CH₂)₄—, —(CH₂)₅—, —(CH₂)₆—, —(CH₂)₇—, —(CH₂)₈—, —(CH₂)₉—, and —(CH₂)₁₀—. In particular embodiments, R¹ can be —(CH₂)₄—, —(CH₂)₆—, or —(CH₂)₈—.

In certain embodiments, R¹ can be —(CH₂)₈—, Y can be C(R³)₂R⁵, each R³ can be H, and R⁵ can be —O(C₁-C₄ alkyl), wherein the C₁-C₄ alkyl can be methyl, ethyl, n-propyl, isopropyl, butyl, sec-butyl, or t-butyl. In particular embodiments, R⁵ can be —OCH₃.

In other embodiments, R¹ can be —(CH₂)₆— and Y can be NR³R⁴. In these embodiments, R³ can be H or C₁-C₆ alkyl and the C₁-C₆ alkyl can be linear, branched, or cyclic. In particular embodiments, the C₁-C₆ alkyl is linear. In still further embodiments, the C₁-C₆ alkyl is CH₃. In other embodiments, R³ is H.

In certain embodiments, R⁴ can be

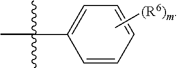

In particular embodiments, the phenyl ring can be substituted m times with R⁶. In some embodiments, m can be 0, 1, 2, 3, 4, or 5. In particular embodiments, m can be 2 or 3.

In embodiments wherein m is 2 or 3, each R⁶ can be, independently at each occurrence, NO₂, N₃, optionally substituted heteroaryl, optionally substituted heterocyclyl,

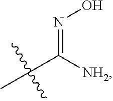 [SMILES: */C(N)=N/O]

—(CH₂)ₚN(R⁷)₂,

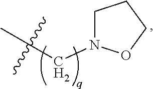

### I, Br, Cl, or F.

In certain embodiments wherein m is 3, the phenyl ring can be 2, 4, 6 substituted with R⁶. In particular embodiments, each R⁶ can be a halogen. And in certain embodiments, each halogen can be a fluorine.

In other embodiments, m can be 2. In such embodiments the phenyl ring can be 2,3-substituted with R⁶, 2,4-substituted with R⁶, 2,5-substituted with R⁶, 2,6-substituted with R⁶, 3,4-substituted with R⁶, 3,5-substituted with R⁶, or 3,6-substituted with R⁶.

In certain embodiments, each R⁶ can be the same. In other embodiments, each R⁶ can be different. In certain embodiments, wherein each R⁶ is different, at least one R⁶ can be NO₂. In such embodiments, the other instance of R⁶ can be N₃, 2H-1,2,3-triazol-2-yl,

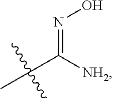 [SMILES: */C(N)=N/O]

1H-tetrazol-1-yl, 2H-tetrazol-2-yl, 1H-pyrazol-1-yl, optionally substituted heterocyclyl, —(CH₂)ₚN(R⁷)₂, or

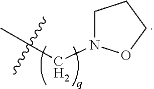

In certain embodiments, the optionally substituted heterocyclyl can be an optionally substituted morpholinyl, an optionally substituted pipiridinyl, optionally substituted piperazinyl, or optionally substituted pyrollidinyl. In particular embodiments, the optionally substituted morpholinyl can be morpholin-4-yl. In still other embodiments, the optionally substituted piperazinyl group can be piperazin-1-yl.

In certain embodiments, the other instance of R⁶ can be —(CH₂)ₚN(R⁷)₂. In some embodiments, p can be 1, 2, or 3. In certain embodiments, p can be 1.

In certain embodiments, each R⁷ can be, independently at each occurrence, H or C₁-C₆ alkyl, or, when taken together along with the nitrogen to which they are bound, is the group

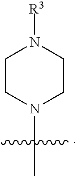

In certain embodiments, each R⁷ can be C₁-C₆ alkyl. In particular embodiments, each R⁷ alkyl can be methyl, ethyl, n-propyl, isopropyl, n-butyl, sec-butyl, or t-butyl. In still further embodiments, each R⁷ can be methyl.

In another embodiment, each R⁷, together with the nitrogen to which they are bound can be

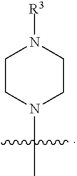

In certain embodiments, the R³ bound to this group can be hydrogen. In other embodiments, the R³ bound to this group can be C₁-C₆ alkyl. In some embodiments, R³ can be methyl, ethyl, n-propyl, isopropyl, n-butyl, sec-butyl, or t-butyl. In still further embodiments, the R³ can be methyl.

In yet another embodiment, m can be 1. In such embodiments, R⁶ can be optionally substituted heteroaryl. In certain embodiments, the optionally substituted heteroaryl can be an optionally substituted pyridinyl or an optionally substituted pyrimidinyl. In some embodiments, the optionally substituted heteroaryl can be an optionally substituted pyrimidinyl. In other embodiments, the optionally substituted heteroaryl can be pyrimidin-2-yl or 2H-tetrazol-2-yl.

In other embodiments, R¹ can be —(CH₂)₈—. In certain embodiments, Y can be optionally substituted heterocyclyl or C(R³)₂R⁵. In some embodiments, Y can be optionally substituted heterocyclyl. In particular embodiments, the optionally substituted heterocyclyl can be selected from the group consisting of aziridinyl, azetidinyl, pyrrolidinyl, imidazolidinyl, pyrazolidinyl, thiazolidinyl, tetrahydrothiophenyl, tetrahydrofuranyl, dioxolyl, pyrrolinyl, piperidyl, piperazinyl, morpholinyl, thiomorpholinyl, tetrahydropyranyl, and tetrahydrothiopyranyl.

In a some embodiments, the optionally substituted heterocyclyl group can be a an optionally substituted tetrahydrofuranyl group. In still another embodiment, the optionally substituted heterocyclyl group can be tetrahydrofuran-2-yl.

In some embodiments, Y can be C(R³)₂R⁵. In certain embodiments, R³ can be, independently at each occurrence, H or C₁-C₆ alkyl. In some embodiments, each R³ is the same. In other embodiments, each R³ is different. In certain embodiments, each R³ is the same and each is a C₁-C₆ alkyl. In particular embodiments, each R³ can be methyl, ethyl, n-propyl, isopropyl, n-butyl, sec-butyl, or t-butyl. In still further embodiments, each R³ can be methyl.

In certain embodiments, R⁵ can be —O(C₁-C₄ alkyl). In certain embodiments, R⁵ can be methoxy, ethoxy, n-propoxy, isopropoxy, n-butoxy, secbutoxy, or tetrbutoxy. In particular embodiments, R⁵ can be methoxy.

In still further embodiments, R¹ can be —(CH₂)₄—. In certain embodiments, Y can NR³R⁴. In certain embodiments, R³ can be H or C₁-C₆ alkyl and the C₁-C₆ alkyl can be linear, branched, or cyclic. In particular embodiments, the C₁-C₆ alkyl is linear. In still further embodiments, the C₁-C₆ alkyl is CH₃. In other embodiments, R³ is H.

In certain embodiments, R⁴ can be

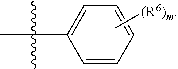

In particular embodiments, the phenyl ring substituted m times with R⁶. In some embodiments, m can be 0, 1, 2, 3, 4, or 5. In particular embodiments, m can be 2 or 3.

In embodiments, wherein m is 2 or 3, each R⁶ can be, independently at each occurrence, NO₂, N₃, optionally substituted heteroaryl, optionally substituted heterocyclyl,

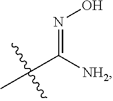 [SMILES: */C(N)=N/O]

—(CH₂)ₚN(R⁷)₂,

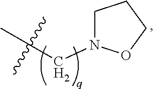

### I, Br, Cl, or F.

In embodiments wherein m is 3, the phenyl ring can be 2, 4, 6 substituted with R⁶. In particular embodiments, each R⁶ can be a halogen. And in even further embodiments, each halogen can be a fluorine.

In other embodiments, m can be 2. In such embodiments the phenyl ring can be 2,3-substituted with R⁶, 2,4-substituted with R⁶, 2,5-substituted with R⁶, 2,6-substituted with R⁶, 3,4-substituted with R⁶, 3,5-substituted with R⁶, or 3,6-substituted with R⁶.

In certain embodiments, each R⁶ can be the same. In other embodiments, each R⁶ can be different. In certain embodiments, wherein each R⁶ is different, at least one R⁶ can be NO₂. In such embodiments, the other instance of R⁶ can be N₃,

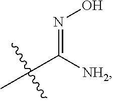 [SMILES: */C(N)=N/O]

optionally substituted heteroaryl, optionally substituted heterocyclyl, —(CH₂)ₚN(R⁷)₂, or

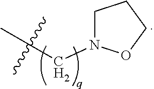

In certain embodiments, the other instance of R⁶ can be

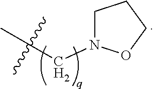

In some embodiments, q can be 1, 2, or 3. In further embodiments, q can be 1.

In certain embodiments, the other instance of R⁶ can be —(CH₂)ₚN(R⁷)₂. In some embodiments, p can be 1, 2, or 3. In further embodiments, p can be 1.

In certain embodiments, each R⁷ can be, independently at each occurrence, H or C₁-C₆ alkyl, or, when taken together along with the nitrogen to which they are bound, is the group

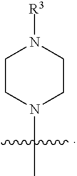

In certain embodiments, each R⁷ can be C₁-C₆ alkyl. In particular embodiments, each R⁷ alkyl can be methyl, ethyl, n-propyl, isopropyl, n-butyl, sec-butyl, or t-butyl. In still further embodiments, each R⁷ can be methyl.

In another embodiment, each R⁷, together with the nitrogen to which they are bound can be

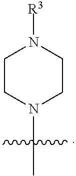

In certain embodiments, the R³ bound to this group can be hydrogen. In other embodiments, the R³ bound to this group can be C₁-C₆ alkyl. In some embodiments, R³ can be methyl, ethyl, n-propyl, isopropyl, n-butyl, sec-butyl, or t-butyl. In still further embodiments, the R³ can be methyl.

In each of the various embodiments described herein, each R² can be hydrogen.

In particular embodiments, the compound of Formula I or II can be a compound selected from Table 1, or a pharmaceutically acceptable salt thereof.

TABLE 1

| Cmpd | | |
|---|---|---|
| # | Structure | Name |
| UV-4 | 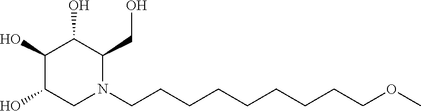 [SMILES: COCCCCCCCCCN1C[C@H](O)[C@@H](O)[C@H](O)[C@H]1CO] | (2R,3R,4R,5S)-2- (hydroxymethyl)-1-(9- methoxynonyl)piperidine-3,4,5- triol (UV-4) |
| UV-5 | 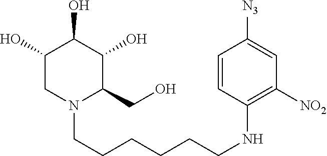 [SMILES: [N-]=[N+]=Nc1ccc(NCCCCCCN2C[C@H](O)[C@@H](O)[C@H](O)[C@H]2CO)c([N+](=O)[O-])c1] | (2R,3R,4R,5S)-1-(6-((4-azido-2- nitrophenyl)amino)hexyl)-2- (hydroxymethyl)piperidine-3,4,5- triol |
| 1 | 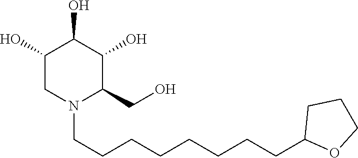 [SMILES: OC[C@@H]1[C@@H](O)[C@H](O)[C@@H](O)CN1CCCCCCCCC1CCCO1] | (2R,3R,4R,5S)-2- (hydroxymethyl)-1-(8- (tetrahydrofuran-2- yl)octyl)piperidine-3,4,5-triol |
| 2 | 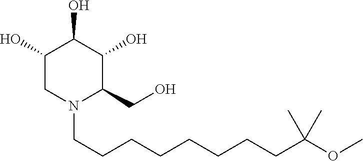 [SMILES: COC(C)(C)CCCCCCCCN1C[C@H](O)[C@@H](O)[C@H](O)[C@H]1CO] | (2R,3R,4R,5S)-2- (hydroxymethyl)-1-(9-methoxy-9- methyldecyl)piperidine-3,4,5-triol |
| 3 | 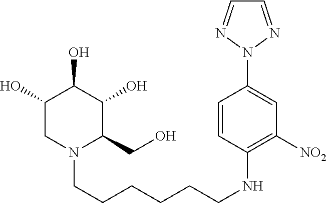 [SMILES: O=[N+]([O-])c1cc(-n2nccn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (2H-1,2,3-triazol-2- yl)phenyl)amino)hexyl)piperidine- 3,4,5-triol |
| 4 | 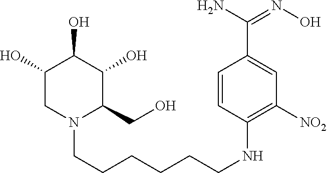 [SMILES: N/C(=N/O)c1ccc(NCCCCCCN2C[C@H](O)[C@@H](O)[C@H](O)[C@H]2CO)c([N+](=O)[O-])c1] | (E)-N′-hydroxy-3-nitro-4-((6- ((2R,3R,4R,5S)-3,4,5-trihydroxy- 2-(hydroxymethyl)piperidin-1- yl)hexyl)amino)benzimidamide |
| 5 | 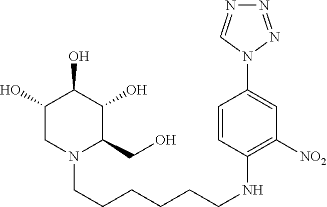 [SMILES: O=[N+]([O-])c1cc(-n2cnnn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (1H-tetrazol-1- yl)phenyl)amino)hexyl)piperidine- 3,4,5-triol |
| 6 | 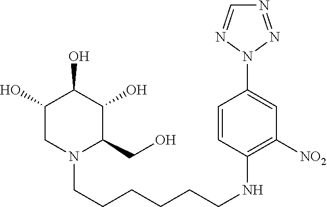 [SMILES: O=[N+]([O-])c1cc(-n2ncnn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (2H-tetrazol-2- yl)phenyl)amino)hexyl)piperidine- 3,4,5-triol |
| 7 | 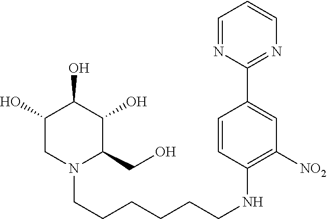 [SMILES: O=[N+]([O-])c1cc(-c2ncccn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (pyrimidin-2- yl)phenyl)amino)hexyl)piperidine- 3,4,5-triol |
| 8 | 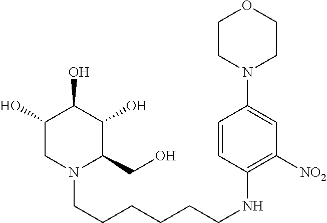 [SMILES: O=[N+]([O-])c1cc(N2CCOCC2)ccc1NCCCCCCN1C[C@H](O)[C@@H](O)[C@H](O)[C@H]1CO] | (2R,3R,4R,5S)-2- (hydroxymethyl)-1-(6-((4- morpholino-2- nitrophenyl)amino)hexyl) piperidine-3,4,5-triol |
| 9 | 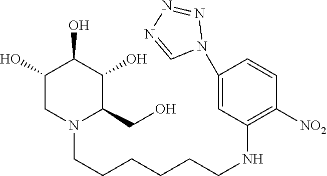 [SMILES: O=[N+]([O-])c1ccc(-n2cnnn2)cc1NCCCCCCN1C[C@H](O)[C@@H](O)[C@H](O)[C@H]1CO] | (2R,3R,4R,5S)-2- (hydroxymethyl)-1-(6-((2-nitro-5- (1H-tetrazol-1- yl)phenyl)amino)hexyl)piperidine- 3,4,5-triol |
| 10 | 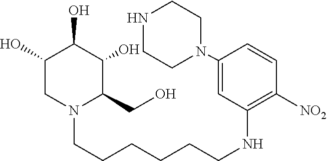 [SMILES: O=[N+]([O-])c1ccc(N2CCNCC2)cc1NCCCCCCN1C[C@H](O)[C@@H](O)[C@H](O)[C@H]1CO] | (2R,3R,4R,5S)-2- (hydroxymethyl)-1-(6-((2-nitro-5- (piperazin-1- yl)phenyl)amino)hexyl)piperidine- 3,4,5-triol |
| 11 | 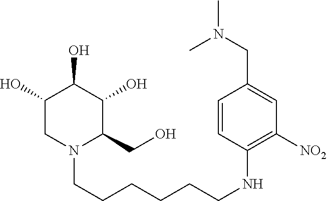 [SMILES: CN(C)Cc1ccc(NCCCCCCN2C[C@H](O)[C@@H](O)[C@H](O)[C@H]2CO)c([N+](=O)[O-])c1] | (2R,3R,4R,5S)-1-(6-((4- ((dimethylamino)methyl)-2- nitrophenyl)amino)hexyl)-2- (hydroxymethyl)piperidine-3,4,5- triol |
| 12 | 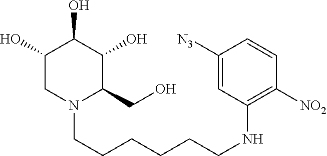 [SMILES: [N-]=[N+]=Nc1ccc([N+](=O)[O-])c(NCCCCCCN2C[C@H](O)[C@@H](O)[C@H](O)[C@H]2CO)c1] | (2R,3R,4R,5S)-1-(6-((5-azido-2- nitrophenyl)amino)hexyl)-2- (hydroxymethyl)piperidine-3,4,5- triol |
| 13 | 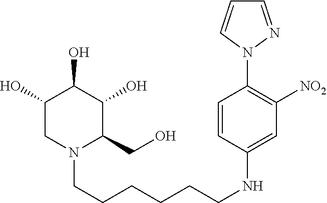 [SMILES: O=[N+]([O-])c1cc(NCCCCCCN2C[C@H](O)[C@@H](O)[C@H](O)[C@H]2CO)ccc1-n1cccn1] | (2R,3R,4R,5S)-2- (hydroxymethyl)-1-(6-((3-nitro-4- (1H-pyrazol-1- yl)phenyl)amino)hexyl)piperidine- 3,4,5-triol |
| 14 | 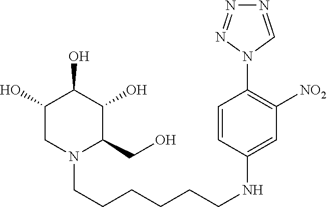 [SMILES: O=[N+]([O-])c1cc(NCCCCCCN2C[C@H](O)[C@@H](O)[C@H](O)[C@H]2CO)ccc1-n1cnnn1] | (2R,3R,4R,5S)-2- (hydroxymethyl)-1-(6-((3-nitro-4- (1H-tetrazol-1- yl)phenyl)amino)hexyl)piperidine- 3,4,5-triol |
| 15 | 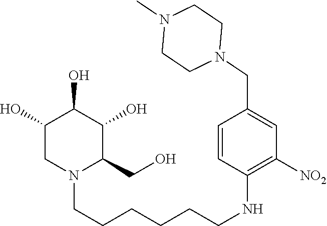 [SMILES: CN1CCN(Cc2ccc(NCCCCCCN3C[C@H](O)[C@@H](O)[C@H](O)[C@H]3CO)c([N+](=O)[O-])c2)CC1] | (2R,3R,4R,5S)-2- (hydroxymethyl)-1-(6-((4-((4- methylpiperazin-1-yl)methyl)-2- nitrophenyl)amino)hexyl) piperidine-3,4,5-triol |
| 16 | 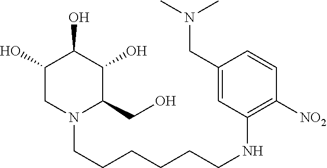 [SMILES: CN(C)Cc1ccc([N+](=O)[O-])c(NCCCCCCN2C[C@H](O)[C@@H](O)[C@H](O)[C@H]2CO)c1] | (2R,3R,4R,5S)-1-(6-((5- ((dimethylamino)methyl)-2- nitrophenyl)amino)hexyl)-2- (hydroxymethyl)piperidine-3,4,5- triol |
| 17 | 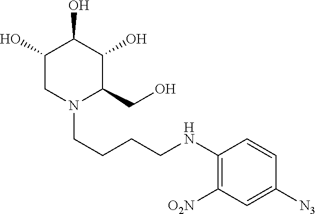 [SMILES: [N-]=[N+]=Nc1ccc(NCCCCN2C[C@H](O)[C@@H](O)[C@H](O)[C@H]2CO)c([N+](=O)[O-])c1] | (2R,3R,4R,5S)-1-(4-((4-azido-2- nitrophenyl)amino)butyl)-2- (hydroxymethyl)piperidine-3,4,5- triol |
| 18 | 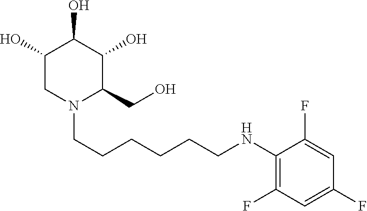 [SMILES: OC[C@@H]1[C@@H](O)[C@H](O)[C@@H](O)CN1CCCCCCNc1c(F)cc(F)cc1F] | (2R,3R,4R,5S)-2- (hydroxymethyl)-1-(6-((2,4,6- trifluorophenyl)amino)hexyl) piperidine- 3,4,5-triol trifluoroacetic acid |
| UV-1 | 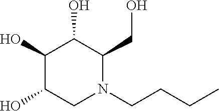 | (2R,3R,4R,5S)-1-butyl -2- (hydroxymethyl)piperidine-3,4,5- triol |
| UV-2 | 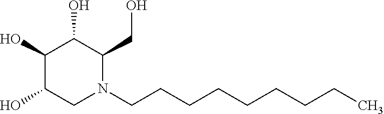 [SMILES: CCCCCCCCCN1C[C@H](O)[C@@H](O)[C@H](O)[C@H]1CO] | (2R,3R,4R,5S)-2- (hydroxymethyl)-1- nonylpiperidine-3,4,5-triol |
| UV-3 | 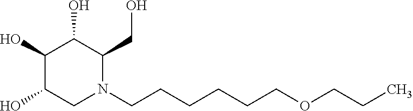 [SMILES: CCCOCCCCCCN1C[C@H](O)[C@@H](O)[C@H](O)[C@H]1CO] | (2R,3R,4R,5S)-2- (hydroxymethyl)-1-(6- propoxyhexyl)piperidine-3,4,5- triol |
| 19 | 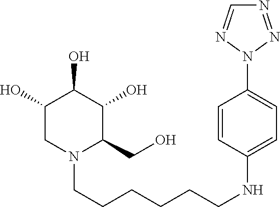 [SMILES: OC[C@@H]1[C@@H](O)[C@H](O)[C@@H](O)CN1CCCCCCNc1ccc(-n2ncnn2)cc1] | (2R,3R,4R,5S)-1-(6-((4-(2H- tetrazol-2-yl)phenyl)amino)hexyl)- 2-(hydroxymethyl)piperidine- 3,4,5-triol |
| 20 | 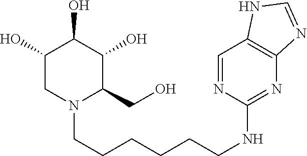 [SMILES: OC[C@@H]1[C@@H](O)[C@H](O)[C@@H](O)CN1CCCCCCNc1ncc2[nH]cnc2n1] | (2R,3R,4R,5S)-1-(6-((7H-purin-2- yl)amino)hexyl)-2- (hydroxymethyl)piperidine-3,4,5- triol |
| 21 | 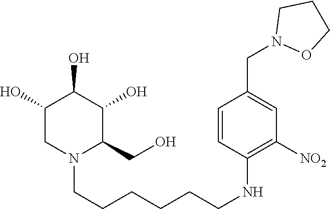 [SMILES: O=[N+]([O-])c1cc(CN2CCCO2)ccc1NCCCCCCN1C[C@H](O)[C@@H](O)[C@H](O)[C@H]1CO] | (2R,3R,4R,5S)-2- (hydroxymethyl)-1-(6-((4- (isoxazolidin-2-ylmethyl)-2- nitrophenyl)amino)hexyl) piperidine-3,4,5-triol |
| 22 | 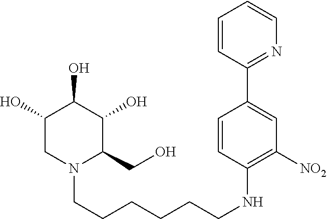 [SMILES: O=[N+]([O-])c1cc(-c2ccccn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (pyridin-2- yl)phenyl)amino)hexyl)piperidine- 3,4,5-triol |
| 23 | 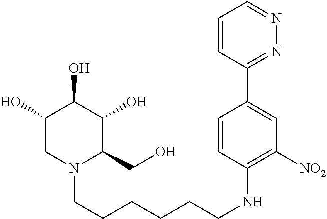 [SMILES: O=[N+]([O-])c1cc(-c2cccnn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (pyridazin-2- yl)phenyl)amino)hexyl)piperidine- 3,4,5-triol |
| 24 | 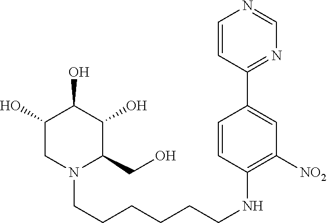 [SMILES: O=[N+]([O-])c1cc(-c2ccncn2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (pyrimidin-4- yl)phenyl)amino)hexyl)piperidine- 3,4,5-triol |
| 25 | 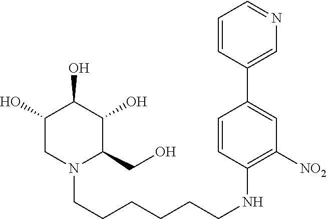 [SMILES: O=[N+]([O-])c1cc(-c2cccnc2)ccc1NCCCCCCN1C[C@H](O)[C@@H](O)[C@H](O)[C@H]1CO] | (2R,3R,4R,5S)-2- (hydroxymethyl)-1-(6-((2-nitro-4- (pyridin-3- yl)phenyl)amino)hexyl)piperidine- 3,4,5-triol |

Salts, hydrates, and solvates of the compounds disclosed herein can also be used in the methods of treating Zika virus disclosed herein. Similarly, all possible stereoisomers and geometric isomers of the compounds disclosed herein, including racemic mixtures and optically pure compounds can be used for treating Zika virus and such compounds are within the scope of this disclosure. When an optically pure compound is desired, it can be obtained, for example, by resolution of a mixture of optical isomers or by stereospecific synthesis from either isomerically pure starting material or use of a chiral auxiliary reagent, for example, see Z. Ma et al., Tetrahedron: Asymmetry, 8(6), pages 883-888 (1997). Resolution of a given compound, an intermediate, or a starting material can be achieved by any suitable method known in the art.

The disclosed compounds can be administered by any suitable route, for example by oral, buccal, inhalation, sublingual, rectal, vaginal, intracisternal, or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique. In particular embodiments, the compounds disclosed herein can be administered orally or intravenously. In other embodiments, the compounds can be administered orally.

For treating a Zika virus infection, the compounds described herein can be administered to a human in an amount ranging from about 1 mg/kg per dose to about 1,000 mg/kg per dose, and in particular embodiments from about 75 mg/kg per dose to about 750 mg/kg per dose. In certain embodiments, the amount of compound described herein that can be given to a human subject in need thereof can be from about 1 mg to about 1000 mg per dose and/or per day.

In certain embodiments, a compound disclosed herein, or a pharmaceutically acceptable salt thereof, can be formulated for the desired route of administration. For example, and in certain embodiments, a compound described herein, or its pharmaceutically acceptable salt, can be formulated for delivery as solution or suspension. In such embodiments, the compound can be dissolved or suspended in water, aqueous saline, aqueous dextrose, or other pharmaceutically acceptable vehicle. In alternative embodiments, the compounds disclosed herein can be formulated in a tablet, capsule, or powdered form.

In certain embodiments of the methods disclosed herein, a compound disclosed herein, it's pharmaceutically acceptable salt, a pharmaceutical composition comprising the compound, or a pharmaceutical composition comprising a pharmaceutically acceptable salt of the compound can be administered to a subject in need thereof once a day, twice a day, three times a day, or four times a day. These administrations could be for one day or for multiple days.

In certain embodiments, a compound disclosed herein, or its pharmaceutically acceptable salt, can be administered prophylactically, e.g., up to 48 hours in advance of contemplated exposure to Zika virus.

In other embodiments, a compound disclosed herein, or its pharmaceutically acceptable salt, can be administered up to 72 hours after the appearance of symptoms of Zika virus infection.

The compounds described herein can be prepared according to methods known to those of skill in the art. For example, compounds wherein R⁶ is optionally substituted aryl can be prepared according to the procedures disclosed in PCT/US2015/059110.

The present disclosure also provides methods of inhibiting Zika virus comprising administering to a subject in need thereof an effective amount of a compound disclosed herein, or a pharmaceutically acceptable salt thereof. In particular embodiments, the present disclosure provides a method of inhibiting Zika virus comprising administering to a subject in need thereof an effective amount of a compound of Formula I or Formula II, or a pharmaceutically acceptable salt thereof. In particular embodiments, the compound of Formula I or II can be a compound selected from Table 1, or a pharmaceutically acceptable salt thereof. In certain embodiments, the subject is a human. In certain embodiments, the human is a human female.

The present disclosure further provides methods of inhibiting replication of Zika virus comprising administering to a subject in need thereof an effective amount of a compound disclosed herein, or a pharmaceutically acceptable salt thereof. In particular embodiments, the present disclosure provides a method of inhibiting replication of Zika virus comprising administering to a subject in need thereof an effective amount of a compound of Formula I or Formula II, or a pharmaceutically acceptable salt thereof. In particular embodiments, the compound of Formula I or II can be a compound selected from Table 1, or a pharmaceutically acceptable salt thereof. In certain embodiments, the subject is a human. In certain embodiments, the human is a human female.

The methods described herein are now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### EXAMPLES

### Example 1: Determination of Cellular Cytotoxicity

In vitro cell cytotoxicity (50% cytotoxic concentration, or CC₅₀) of the compounds set forth in Table 2, below, was evaluated in Vero cells. Each compound was serially diluted starting from a maximum concentration of 500 or 1000 μM. Cytotoxicity was determined after 3-5 days using the CellTiter-Glo® kit (Promega) per the manufacturer's directions. Luminescence was read in a Tecan reader. Cytotoxicity was determined using untreated tissue as 0% cytotoxic effect and 20% DMSO-treated tissue as 100% cytotoxic effect.

TABLE 2

| Com- | | |
|---|---|---|
| pound | CC₅₀ (μM) | Compound Name |
| UV-4 | >1000 | (2R,3R,4R,5S)-2-(hydroxymethyl)-1-(9- |
| | | methoxynonyl)piperidine-3,4,5-triol (UV-4) |
| UV-5 | 77.3 | (2R,3R,4R,5S)-1-(6-((4-azido-2- |
| | | nitrophenyl)amino)hexyl)-2- |
| | | (hydroxymethyl)piperidine-3,4,5-triol |
| 1 | >500 | (2R,3R,4R,5S)-2-(hydroxymethyl)-1-(8- |
| | | (tetrahydrofuran-2-yl)octyl)piperidine-3,4,5-triol |
| 2 | >500 | (2R,3R,4R,5S)-2-(hydroxymethyl)-1-(9-methoxy- |
| | | 9-methyldecyl)piperidine-3,4,5-triol |
| 3 | 245 | (2R,3R,4R,5S)-2-(hydroxymethyl)-1-(6-((2-nitro-4- |
| | | (2H-1,2,3-triazol-2- |
| | | yl)phenyl)amino)hexyl)piperidine-3,4,5-triol |
| 4 | >1000 | (E)—N′-hydroxy-3-nitro-4-((6-((2R,3R,4R,5S)- |
| | | 3,4,5-trihydroxy-2-(hydroxymethyl)piperidin-1- |
| | | yl)hexyl)amino)benzimidamide |
| 5 | >1000 | (2R,3R,4R,5S)-2-(hydroxymethyl)-1-(6-((2-nitro-4- |
| | | (1H-tetrazol-1-yl)phenyl)amino)hexyl)piperidine- |
| | | 3,4,5-triol |
| 6 | 952.9 | (2R,3R,4R,5S)-2-(hydroxymethyl)-1-(6-((2-nitro-4- |
| | | (2H-tetrazol-2-yl)phenyl)amino)hexyl)piperidine- |
| | | 3,4,5-triol |
| 7 | >1000 | (2R,3R,4R,5S)-2-(hydroxymethyl)-1-(6-((2-nitro-4- |
| | | (pyrimidin-2-yl)phenyl)amino)hexyl)piperidine- |
| | | 3,4,5-triol |
| 8 | >1000 | (2R,3R,4R,5S)-2-(hydroxymethyl)-1-(6-((4- |
| | | morpholino-2-nitrophenyl)amino)hexyl)piperidine- |
| | | 3,4,5-triol |
| 9 | >1000 | (2R,3R,4R,5S)-2-(hydroxymethyl)-1-(6-((2-nitro-5- |
| | | (1H-tetrazol-1-yl)phenyl)amino)hexyl)piperidine- |
| | | 3,4,5-triol |
| 10 | >1000 | (2R,3R,4R,5S)-2-(hydroxymethyl)-1-(6-((2-nitro-5- |
| | | (piperazin-1-yl)phenyl)amino)hexyl)piperidine- |
| | | 3,4,5-triol |
| 11 | >1000 | (2R,3R,4R,5S)-1-(6-((4-((dimethylamino)methyl)- |
| | | 2-nitrophenyl)amino)hexyl)-2- |
| | | (hydroxymethyl)piperidine-3,4,5-triol |
| 12 | 139.9 | (2R,3R,4R,5S)-1-(6-((5-azido-2- |
| | | nitrophenyl)amino)hexyl)-2- |
| | | (hydroxymethyl)piperidine-3,4,5-triol |
| 13 | >1000 | (2R,3R,4R,5S)-2-(hydroxymethyl)-1-(6-((3-nitro-4- |
| | | (1H-pyrazol-1-yl)phenyl)amino)hexyl)piperidine- |
| | | 3,4,5-triol |
| 14 | >1000 | (2R,3R,4R,5S)-2-(hydroxymethyl)-1-(6-((3-nitro-4- |
| | | (1H-tetrazol-1-yl)phenyl)amino)hexyl)piperidine- |
| | | 3,4,5-triol |
| 15 | >1000 | (2R,3R,4R,5S)-2-(hydroxymethyl)-1-(6-((4-((4- |
| | | methylpiperazin-1-yl)methyl)-2- |
| | | nitrophenyl)amino)hexyl)piperidine-3,4,5-triol |
| 16 | >1000 | (2R,3R,4R,5S)-1-(6-((5-((dimethylamino)methyl)- |
| | | 2-nitrophenyl)amino)hexyl)-2- |
| | | (hydroxymethyl)piperidine-3,4,5-triol |
| 17 | 350.3 | (2R,3R,4R,5S)-1-(4-((4-azido-2- |
| | | nitrophenyl)amino)butyl)-2- |
| | | (hydroxymethyl)piperidine-3,4,5-triol |
| 18 | 413.04 | (2R,3R,4R,5S)-2-(hydroxymethyl)-1-(6-((2,4,6- |
| | | trifluorophenyl)amino)hexyl)piperidine-3,4,5-triol |
| | | trifluoroacetic acid |
| 19 | 952.9 | (2R,3R,4R,5S)-1-(6-((4-(2H-tetrazol-2- |
| | | yl)phenyl)amino)hexyl)-2- |
| | | (hydroxymethyl)piperidine-3,4,5-triol |
| 20 | 831.8 | (2R,3R,4R,5S)-1-(6-((7H-purin-2-yl)amino)hexyl)- |
| | | 2-(hydroxymethyl)piperidine-3,4,5-triol |
| 21 | 2133 | (2R,3R,4R,5S)-2-(hydroxymethyl)-1-(6-((4- |
| | | (isoxazolidin-2-ylmethyl)-2- |
| | | nitrophenyl)amino)hexyl)piperidine-3,4,5-triol |
| 22 | 99.8 | (2R,3R,4R,5S)-2-(hydroxymethyl)-1-(6-((2-nitro-4- |
| | | (pyridin-2-yl)phenyl)amino)hexyl)piperidine-3,4,5- |
| | | triol |
| 23 | 5190 | (2R,3R,4R,5S)-2-(hydroxymethyl)-1-(6-((2-nitro-4- |
| | | (pyridazin-3-yl)phenyl)amino)hexyl)piperidine- |
| | | 3,4,5-triol |
| 24 | 816 | (2R,3R,4R,5S)-2-(hydroxymethyl)-1-(6-((2-nitro-4- |
| | | (pyrimidin-4-yl)phenyl)amino)hexyl)piperidine- |
| | | 3,4,5-triol |
| 25 | 100.8* | (2R,3R,4R,5S)-2-(hydroxymethyl)-1-(6-((2-nitro-4- |
| | | (pyridin-3-yl)phenyl)amino)hexyl)piperidine-3,4,5- |
| | | triol |

*Average of two values

These results clearly show that the compounds disclosed herein were well tolerated by the Vero cells, suggesting low overall cytotoxicity for the compounds disclosed herein.

### Example 2: Efficacy of UV-4 Against Lethal Zika Virus Infection in Mice

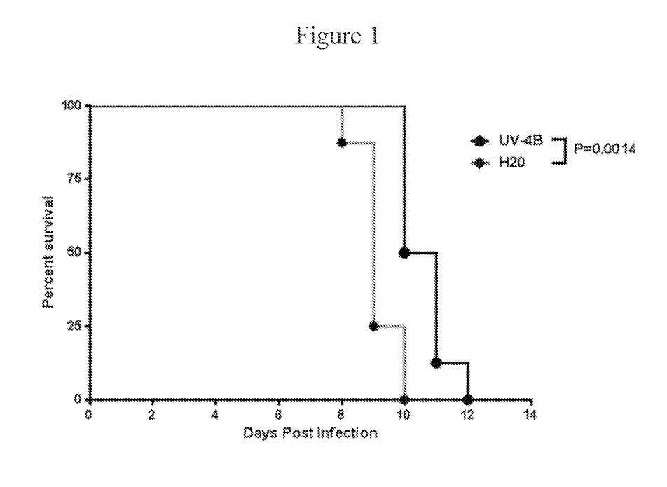

AG129 mice, aged 5-7 weeks, of both sexes were divided into groups of eight mice each. Mice were infected with 1E2 FFU of Zika virus (isolate FSS13025 of the Asian genotype). The mice were treated 16 hours after exposure. One group was administered 750 mg/kg of UV-4 once daily for three days. UV-4 was formulated in water and all doses were administered via oral gavage in a volume of 100 μL. A control group was administered vehicle (water) only. Predefined study endpoints were mortality, euthanasia due to severe morbidity, or termination after 10 days total. Weights were measured and health scores were determined daily. As is shown in FIG. 1, treatment with UV-4 provided a statistically significant survival benefit.

### Example 3: In Vitro Antiviral Activity

The iminosugar compounds set forth in Table 4, below, were evaluated for antiviral activity against Zika virus, FSS 13025 strain (ZIKV, Cambodia 2010) and PR ABC strain (ZIKV, PR2015) in an yield reduction assay followed by a plaque assay.

### Antiviral Activity Against ZIKV, Cambodian 2010

In the yield reduction assay, Vero cells were seeded in 24-well plates and incubated overnight. The next day, serial dilutions of a given compound were prepared at 300 μM, 100 μM, 30 μM, 10 μM, and 3.0 μM. Growth medium was removed from the cells and the serial dilutions of the given compound were added in duplicate for one hour. Zika virus, FSS 13025 strain (ZIKV, Cambodia 2010) was added at a multiplicity of infection (MOI) of 0.01 for one hour. Growth medium was added to 1 mL and the cells were incubated for 4 days. Thereafter, cell supernatants were harvested, cleared of cell debris by centrifugation, and stored at −80° C. until further analysis for viral content in a plaque assay.

Plaque assays were performed by seeding Vero cells in 24-well plates and allowing the cells to adhere overnight. The next day each of the harvested supernatants from the yield-reduction assay were serially diluted 10-fold, growth medium was removed from the cells, and the adhered cells were infected with the dilutions of the supernatants for 1 h. Methylcellulose was added and the cells were incubated for 3 days. Plaques were visualized by staining the cell monolayer with crystal violet. Relative inhibition was calculated using IDBS XLFIT analysis program.

### Antiviral Activity Against ZIKV, PR2015

In the yield reduction assay, Vero cells were seeded in 24-well plates and incubated overnight. The next day, serial dilutions of a given compound were prepared at 200 μM, 100 μM, 20 μM, 4 μM, 0.8 μM, 0.16 μM, 0.03 μM, and 0.01 μM. Growth medium was removed from the cells and the serial dilutions of the given compound were added in duplicate for one hour. Zika virus, PR ABC strain (ZIKV, PR2015) was added at a multiplicity of infection (MOI) of 0.01 for one hour. Growth medium was added to 1 mL and the cells were incubated for 5 days. Thereafter, cell supernatants were harvested, cleared of cell debris by centrifugation, and stored at −80° C. until further analysis for viral content in a plaque assay.

Plaque assays were performed by seeding Vero cells in 24-well plates and allowing the cells to adhere overnight. The next day each of the harvested supernatants from the yield-reduction assay were serially diluted 10-fold, growth medium was removed from the cells, and the adhered cells were infected with the dilutions of the supernatants for 1 h. The cells were incubated with 0.8% carboxymethyl cellulose (CMC) for 3 days. At the end of the incubation, CMC overlay was removed, and the cells were permeabilized with an 80%/20% (V/V) mixture of ethanol and methanol for 10 min at −20° C. Plaques were visualized by performing an immunostaining assay with monoclonal 4G2 (ATCC) which was detected by HRP-conjugated goat anti-mouse antibody and visualized using HRP-conjugated goat anti-mouse polyclonal antibodies. Relative inhibition was calculated using IDBS XLFIT analysis program.

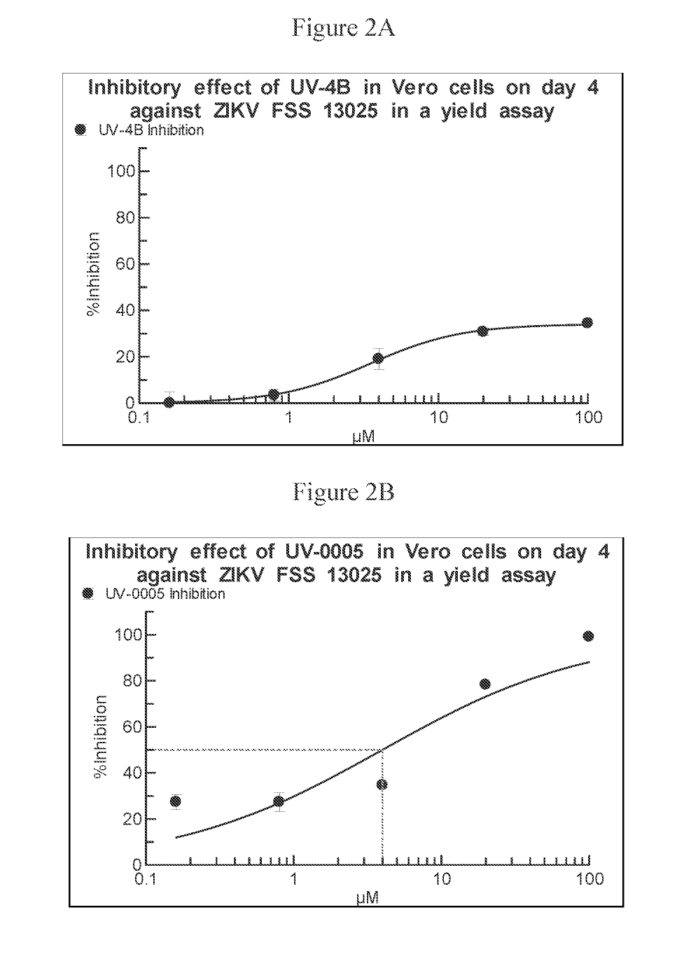

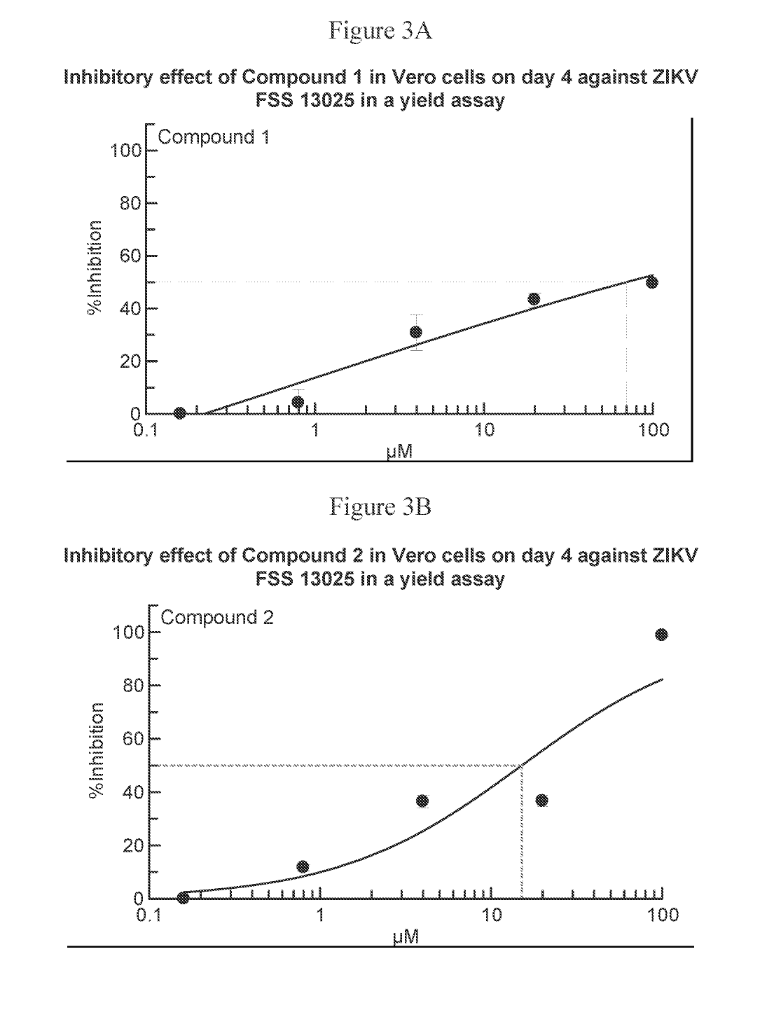

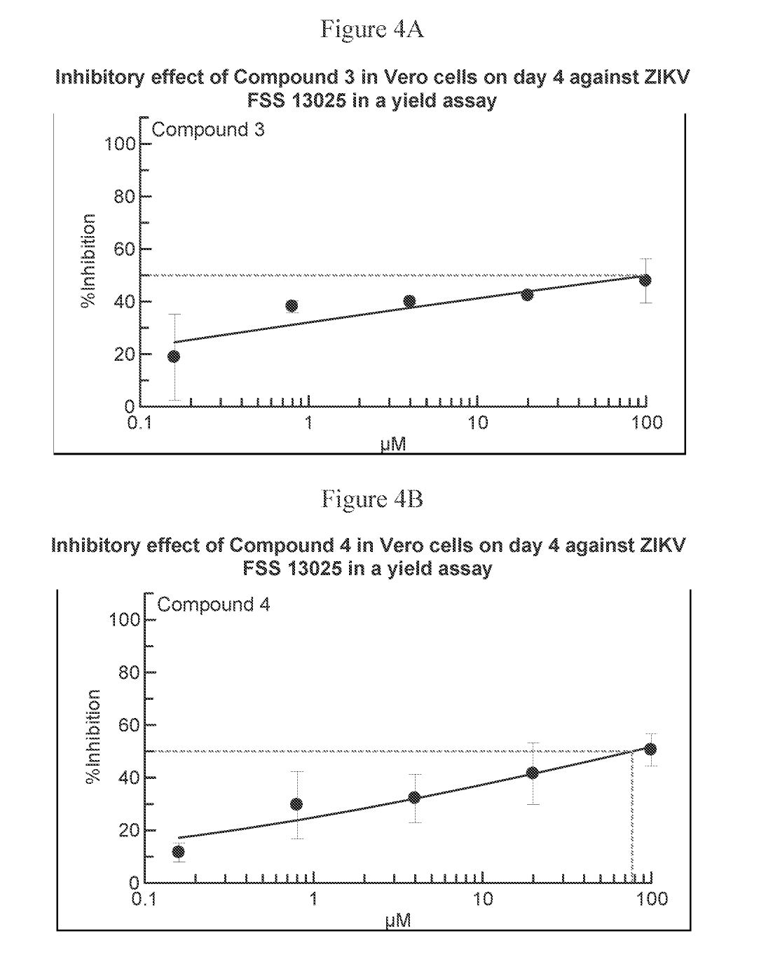

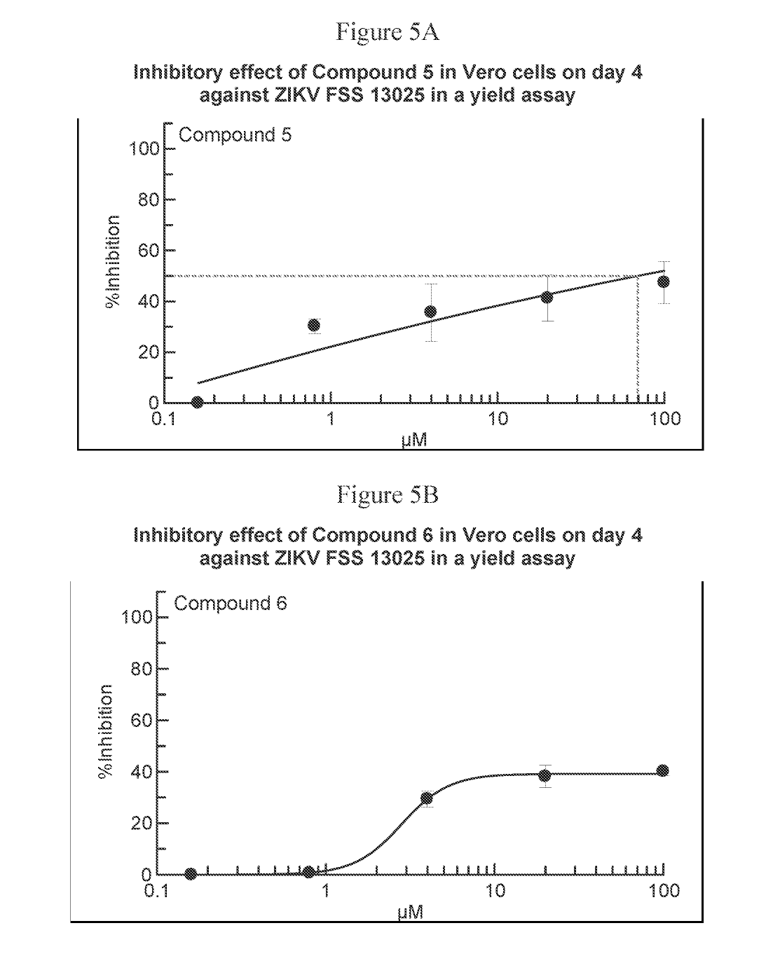

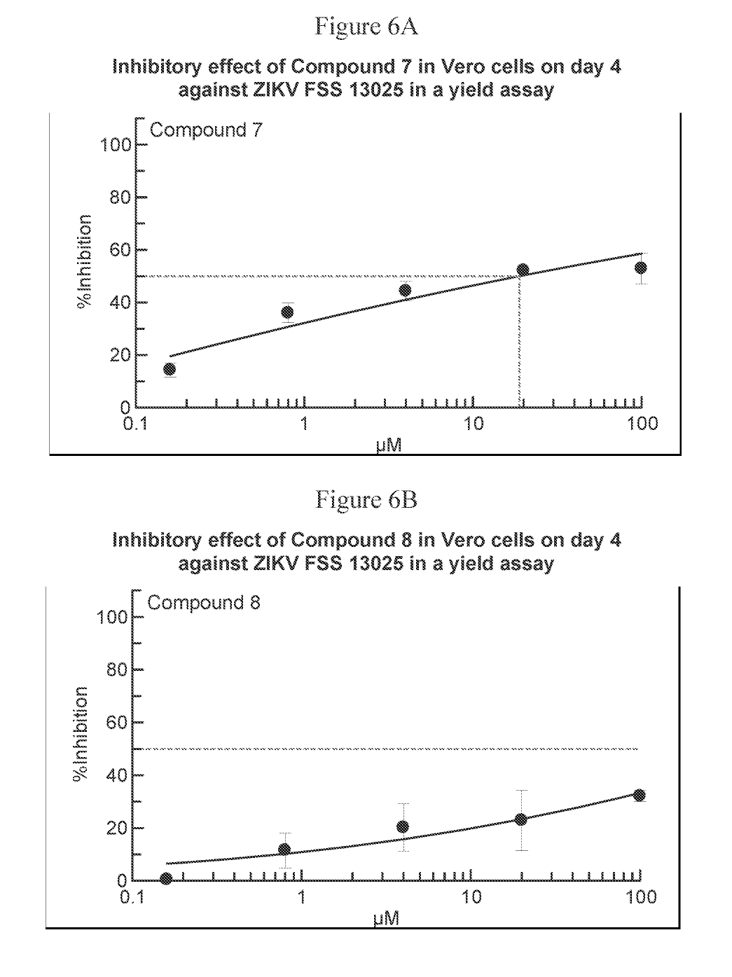

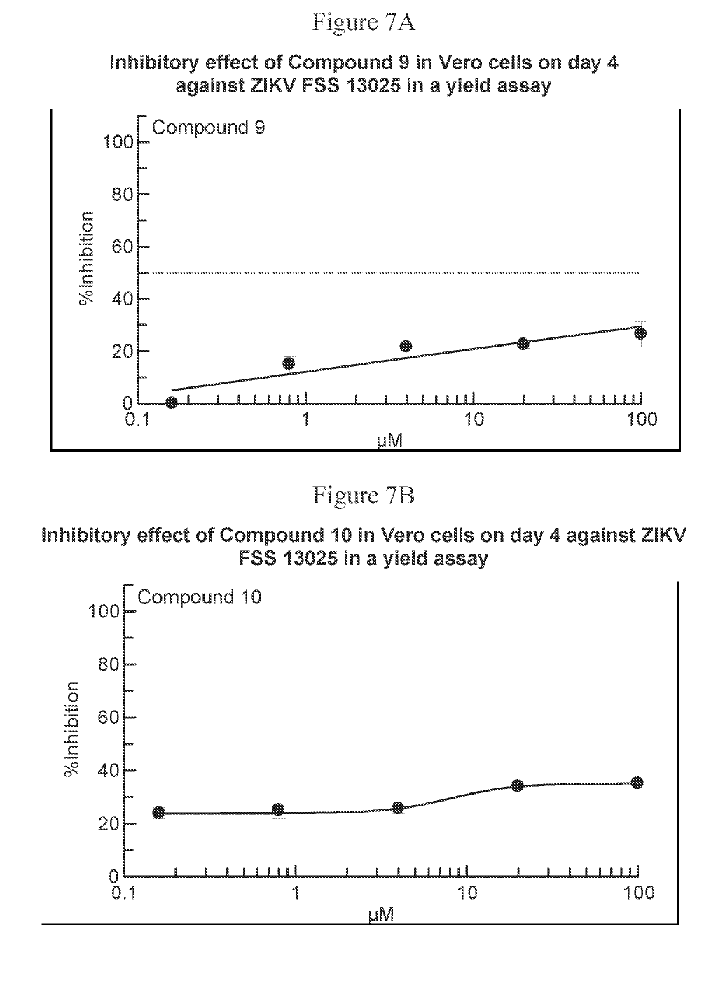

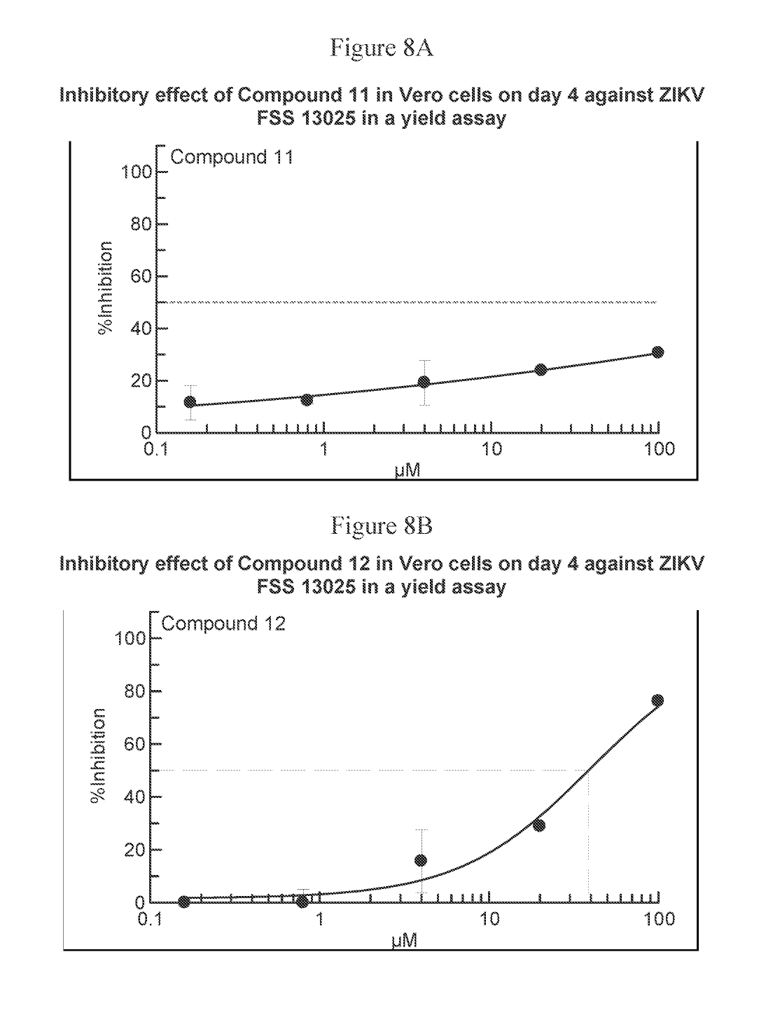

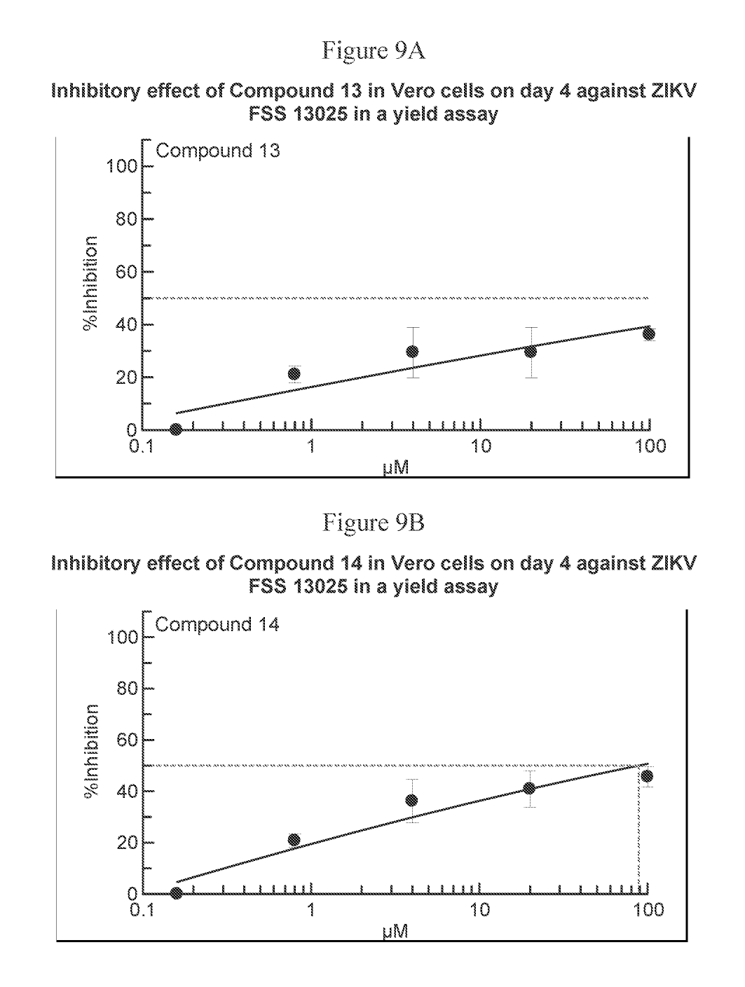

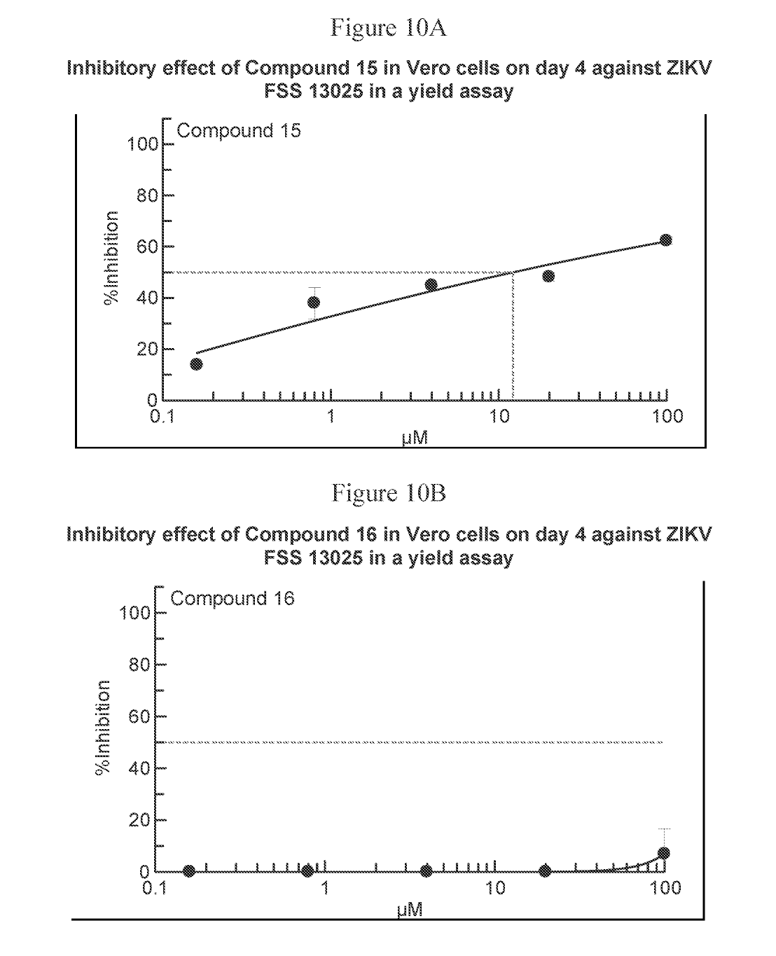

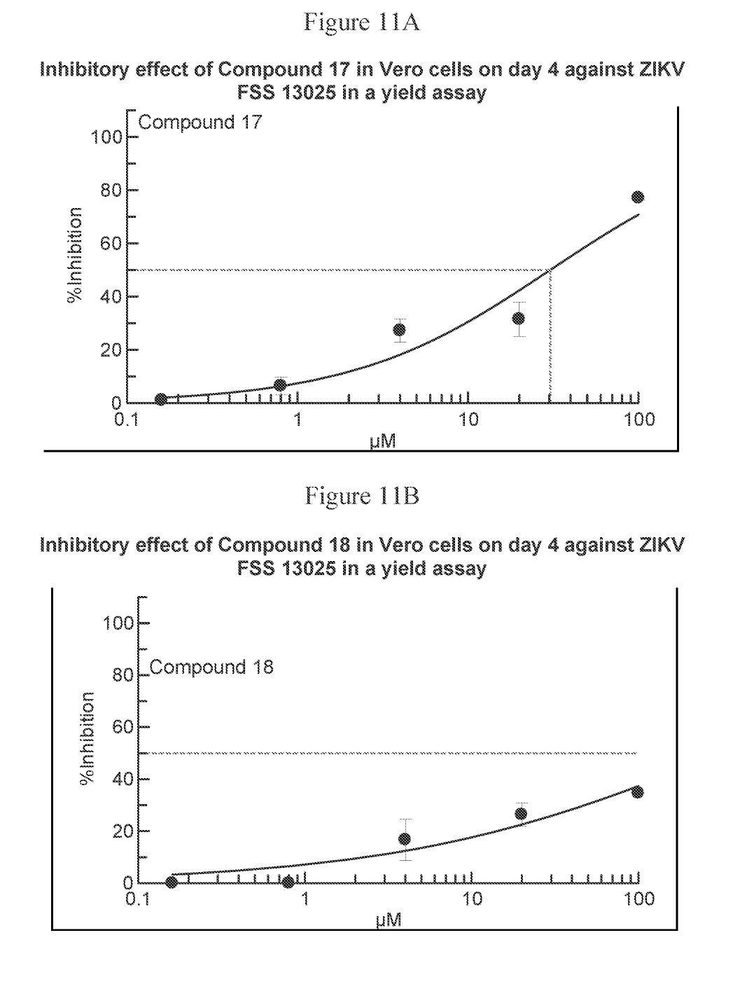

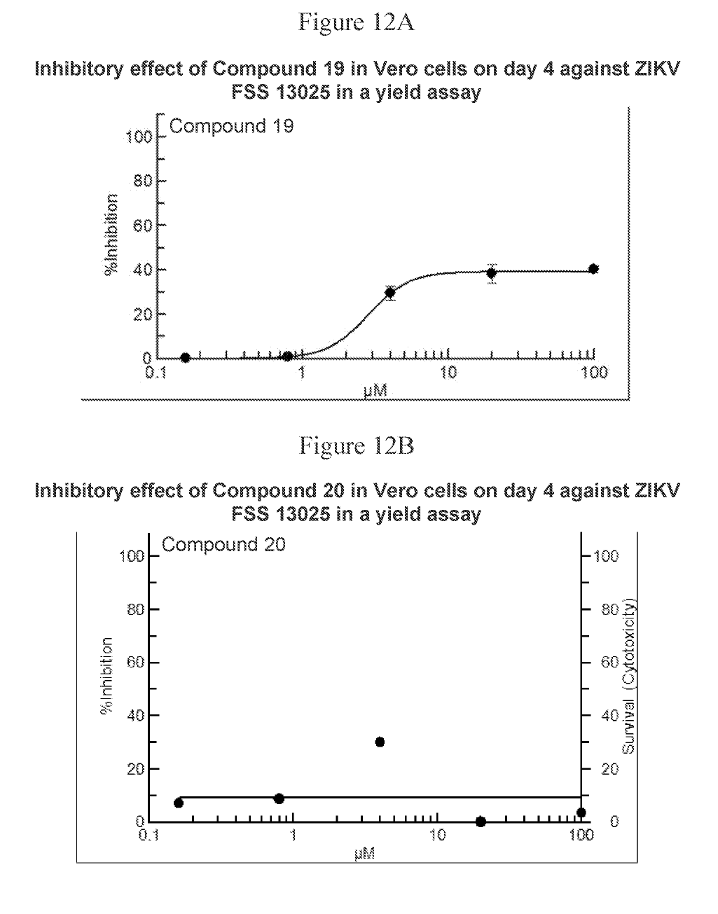

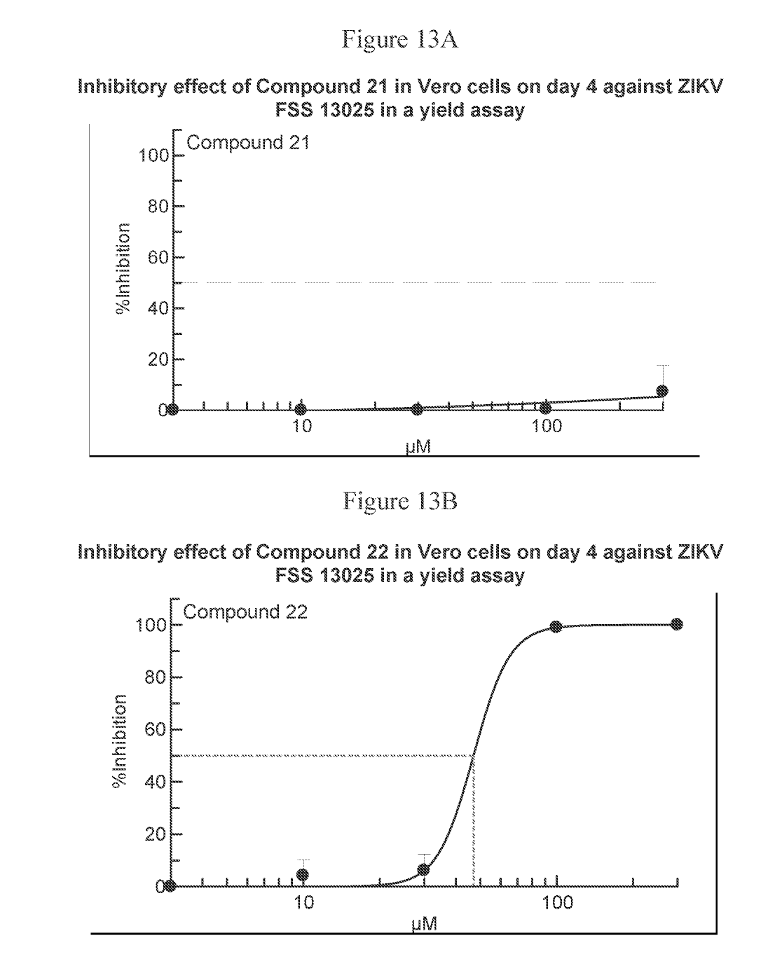

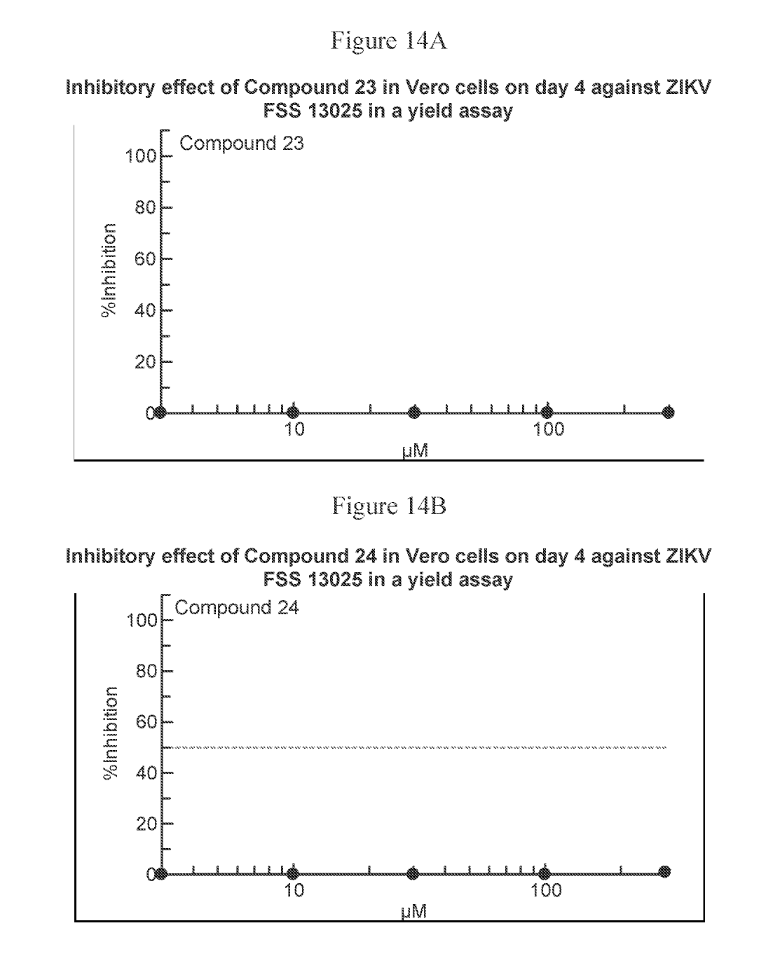

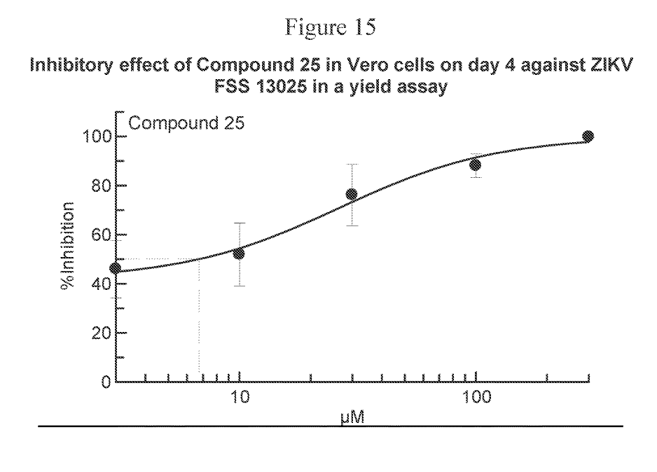

The duplicate titers for virus-only (vehicle control) were averaged and used as 0% inhibition. Efficacy data for the compounds were reported as the mean of the duplicates, including the standard deviation (SD) and coefficient of variation (% CV). FIGS. 2A, 2B, 3A, 3B, 4A, 4B, 5A, 5B, 6A, 6B, 7A, 7B, 8A, 8B, 9A, 9B, 10A, 10B, 11A, 11B, 12A, 12B, 13A, 13B, 14A, 14B, and 15 show the in vitro inhibitory effect of the iminosugar compounds against Zika virus, FSS 13025 strain, as set forth in Table 3 below.

The resulting IC₅₀s are shown in Table 3.

| | ZIKV, | | |
|---|---|---|---|
| | Cambodian | ZIKV, | |
| | 2010 | PR2015 | |
| Compound | IC₅₀ (μM) | IC₅₀ (μM) | Compound Name |
| UV-1 | n.d. | 3 | (2R,3R,4R,5S)-1-butyl-2- |
| | | | (hydroxymethyl)piperidine-3,4,5-triol |
| UV-2 | n.d. | 95 | (2R,3R,4R,5S)-2-(hydroxymethyl)-1- |
| | | | nonylpiperidine-3,4,5-triol |
| UV-3 | n.d. | >100 | (2R,3R,4R,5S)-2-(hydroxymethyl)-1-(6- |
| | | | propoxyhexyl)piperidine-3,4,5-triol |
| UV-4 | >100 | >100 | (2R,3R,4R,5S)-2-(hydroxymethyl)-1-(9- |
| | | | methoxynonyl)piperidine-3,4,5-triol |
| UV-5 | 4.42* | 5.24* | (2R,3R,4R,5S)-1-(6-((4-azido-2- |
| | | | nitrophenyl)amino)hexyl)-2- |
| | | | (hydroxymethyl)piperidine-3,4,5-triol |
| 1 | 45.66* | 69.61 | (2R,3R,4R,5S)-2-(hydroxymethyl)-1-(8- |
| | | | (tetrahydrofuran-2-yl)octyl)piperidine-3,4,5-triol |
| 2 | 10.11* | 12.56* | (2R,3R,4R,5S)-2-(hydroxymethyl)-1-(9- |
| | | | methoxy-9-methyldecyl)piperidine-3,4,5-triol |
| 3 | 105.8 | n.d. | (2R,3R,4R,5S)-2-(hydroxymethyl)-1-(6-((2- |
| | | | nitro-4-(2H-1,2,3-triazol-2- |
| | | | yl)phenyl)amino)hexyl)piperidine-3,4,5-triol |
| 4 | 77.07 | n.d. | (E)—N′-hydroxy-3-nitro-4-((6-((2R,3R,4R,5S)- |
| | | | 3,4,5-trihydroxy-2-(hydroxymethyl)piperidin-1- |
| | | | yl)hexyl)amino)benzimidamide |
| 5 | 69.29 | n.d. | (2R,3R,4R,5S)-2-(hydroxymethyl)-1-(6-((2- |
| | | | nitro-4-(1H-tetrazol-1- |
| | | | yl)phenyl)amino)hexyl)piperidine-3,4,5-triol |
| 7 | 18.86 | n.d. | (2R,3R,4R,5S)-2-(hydroxymethyl)-1-(6-((2- |
| | | | nitro-4-(pyrimidin-2- |
| | | | yl)phenyl)amino)hexyl)piperidine-3,4,5-triol |
| 8 | >100 | >100 | (2R,3R,4R,5S)-2-(hydroxymethyl)-1-(6-((4- |
| | | | morpholino-2- |
| | | | nitrophenyl)amino)hexyl)piperidine-3,4,5-triol |
| 9 | >100 | n.d. | (2R,3R,4R,5S)-2-(hydroxymethyl)-1-(6-((2- |
| | | | nitro-5-(1H-tetrazol-1- |
| | | | yl)phenyl)amino)hexyl)piperidine-3,4,5-triol |
| 10 | >100 | n.d. | (2R,3R,4R,5S)-2-(hydroxymethyl)-1-(6-((2- |
| | | | nitro-5-(piperazin-1- |
| | | | yl)phenyl)amino)hexyl)piperidine-3,4,5-triol |
| 11 | >100 | >100 | (2R,3R,4R,5S)-1-(6-((4- |
| | | | ((dimethylamino)methyl)-2- |
| | | | nitrophenyl)amino)hexyl)-2- |
| | | | (hydroxymethyl)piperidine-3,4,5-triol |
| 12 | 38.71 | 94.6 | (2R,3R,4R,5S)-1-(6-((5-azido-2- |
| | | | nitrophenyl)amino)hexyl)-2- |
| | | | (hydroxymethyl)piperidine-3,4,5-triol |
| 13 | >100 | n.d. | (2R,3R,4R,5S)-2-(hydroxymethyl)-1-(6-((3- |
| | | | nitro-4-(1H-pyrazol-1- |
| | | | yl)phenyl)amino)hexyl)piperidine-3,4,5-triol |
| 14 | 88.45 | n.d. | (2R,3R,4R,5S)-2-(hydroxymethyl)-1-(6-((3- |
| | | | nitro-4-(1H-tetrazol-1- |
| | | | yl)phenyl)amino)hexyl)piperidine-3,4,5-triol |
| 15 | 12.14 | 150 | (2R,3R,4R,5S)-2-(hydroxymethyl)-1-(6-((4-((4- |
| | | | methylpiperazin-1-yl)methyl)-2- |
| | | | nitrophenyl)amino)hexyl)piperidine-3,4,5-triol |
| 16 | >100 | n.d. | (2R,3R,4R,5S)-1-(6-((5- |
| | | | ((dimethylamino)methyl)-2- |
| | | | nitrophenyl)amino)hexyl)-2- |
| | | | (hydroxymethyl)piperidine-3,4,5-triol |
| 17 | 30.36 | n.d. | (2R,3R,4R,5S)-1-(4-((4-azido-2- |
| | | | nitrophenyl)amino)butyl)-2- |
| | | | (hydroxymethyl)piperidine-3,4,5-triol |
| 18 | >100 | n.d. | (2R,3R,4R,5S)-2-(hydroxymethyl)-1-(6-((2,4,6- |
| | | | trifluorophenyl)amino)hexyl)piperidine-3,4,5- |
| | | | triol trifluoroacetic acid |
| 19 | 140.3 | n.d. | (2R,3R,4R,5S)-1-(6-((4-(2H-tetrazol-2- |
| | | | yl)phenyl)amino)hexyl)-2- |
| | | | (hydroxymethyl)piperidine-3,4,5-triol |
| 20 | n.d. | >100 | (2R,3R,4R,5S)-1-(6-((7H-purin-2- |
| | | | yl)amino)hexyl)-2-(hydroxymethyl)piperidine- |
| | | | 3,4,5-triol |
| 21 | >100 | n.d. | (2R,3R,4R,5S)-2-(hydroxymethyl)-1-(6-((4- |
| | | | (isoxazolidin-2-ylmethyl)-2- |
| | | | nitrophenyl)amino)hexyl)piperidine-3,4,5-triol |
| 22 | 46.96 | n.d. | (2R,3R,4R,5S)-2-(hydroxymethyl)-1-(6-((2- |
| | | | nitro-4-(pyridin-2- |
| | | | yl)phenyl)amino)hexyl)piperidine-3,4,5-triol |
| 23 | >100 | n.d. | (2R,3R,4R,5S)-2-(hydroxymethyl)-1-(6-((2- |
| | | | nitro-4-(pyridazin-3- |
| | | | yl)phenyl)amino)hexyl)piperidine-3,4,5-triol |
| 24 | >100 | n.d. | (2R,3R,4R,5S)-2-(hydroxymethyl)-1-(6-((2- |
| | | | nitro-4-(pyrimidin-4- |
| | | | yl)phenyl)amino)hexyl)piperidine-3,4,5-triol |
| 25 | 17.01* | n.d. | (2R,3R,4R,5S)-2-(hydroxymethyl)-1-(6-((2- |
| | | | nitro-4-(pyridin-3- |
| | | | yl)phenyl)amino)hexyl)piperidine-3,4,5-triol |

*Efficacy data is an average of multiple data points.
n.d. - IC₅₀ value not determined.

While certain compounds had IC₅₀s exceeding 100 μM, other compounds had low micromolar activity, including compounds UV-5, 2, 7, 12, 15, 17, 22, and 25.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. Also incorporated by reference is any supplemental information that was published along with any of the aforementioned publications, patents and patent applications. For example, some journal articles are published with supplemental information that is typically available online.

What is claimed is:

1. A method of treating a Zika virus infection, the method comprising administering to a subject in need thereof, an effective amount of a compound according to Formula I: 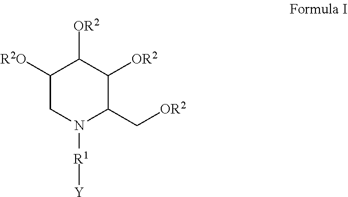
wherein:
R¹ is C₁-C₁₀ alkyl;
each R² is independently H, C₁-C₆ alkyl, or C₁-C₆ perfluoroalkyl;
Y is NR³R⁴, optionally substituted heterocyclyl, or C(R³)₂R⁵;
each R³ is independently H or C₁-C₆ alkyl;
R⁴ is 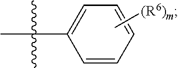
m is 0, 1, 2, 3, 4 or 5;
R⁵ is —O(C₁-C₄ alkyl);
R⁶ is, independently at each occurrence, NO₂, N₃, optionally substituted heteroaryl, optionally substituted heterocyclyl, 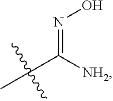 [SMILES: */C(N)=N/O]
—(CH₂)ₚN(R⁷)₂, 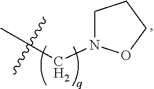
I, Br, Cl, or F;
p is 1, 2, or 3;
q is 1, 2, or 3; and
R⁷ is, independently at each occurrence, H or C₁-C₆ alkyl, or, when taken together along with the nitrogen to which they are bound, is the group 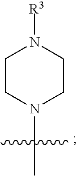
or a pharmaceutically acceptable salt thereof.

2. The method of claim 1, wherein the compound of Formula I is a compound of Formula II:

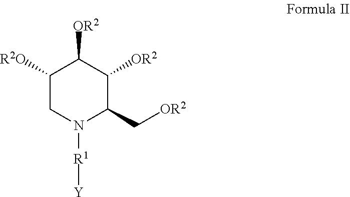

3. The method of claim 2, wherein R¹ is selected from the group consisting of —CH₂, —CH₂CH₂—, —(CH₂)₃—, —(CH₂)₄—, —(CH₂)₅—, —(CH₂)₆—, —(CH₂)₇—, —(CH₂)₈—, —(CH₂)₉—, and —(CH₂)₁₀—.

4. The method of claim 3, wherein R¹ is —(CH₂)₄—, —(CH₂)₆—, or —(CH₂)₈—.

5. The method of claim 4, wherein R¹ is —(CH₂)₈— and Y is C(R³)₂R⁵.

6. The method of claim 5, wherein each R³ is H and R⁵ is —O(C₁-C₄ alkyl), wherein the C₁-C₄ alkyl is selected from the group consisting of methyl, ethyl, n-propyl, isopropyl, butyl, sec-butyl, and t-butyl

7. The method of claim 4, wherein R¹ is —(CH₂)₆— and Y is NR³R⁴.

8. The method of claim 7, wherein R³ is H and R⁴ is

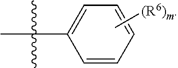

9. The method of claim 7, wherein m is 1, 2, or 3, and R⁶ is independently at each occurrence NO₂, N₃, optionally substituted heteroaryl, optionally substituted heterocyclyl, 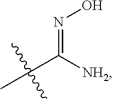 [SMILES: */C(N)=N/O]
—(CH₂)ₚN(R⁷)₂, 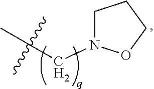
I, Br, Cl, or F.

10. The method of claim 9, wherein m is 3 and each R⁶ is halogen.

11. The method of claim 10, wherein the halogen is fluorine.

12. The method of claim 9, wherein m is 2, at least one R⁶ is NO₂, and a second R⁶ is N₃, 2H-1,2,3-triazol-2-yl, 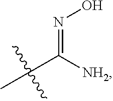 [SMILES: */C(N)=N/O]
1H-tetrazol-1-yl, 1H-pyrazol-1-yl, optionally substituted heterocyclyl, —(CH₂)ₚN(R⁷)₂, or

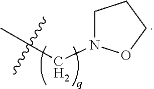

13. The method of claim 9, wherein m is 1 and R⁶ is optionally substituted heteroaryl.

14. The method of claim 9, where in the optionally substituted heteroaryl is pyrimidin-2-yl or 2H-tetrazol-2-yl.

15. The method of claim 4, wherein R¹ is —(CH₂)₈— and Y is optionally substituted heterocyclyl or C(R³)₂R⁵.

16. The method of claim 15, wherein Y is tetrahydrofuran-2-yl.

17. The method of claim 15, wherein Y is C(R³)₂R⁵.

18. The method of claim 17, wherein R³ is independently at each occurrence, H or C₁-C₆ alkyl.

19. The method of claim 18, wherein R³ is independently at each occurrence, methyl, ethyl, n-propyl, isopropyl, n-butyl, sec-butyl, or t-butyl.

20. The method of claim 19, wherein R³ is methyl at each occurrence.

21. The method of claim 20, wherein R⁵ is methoxy.

22. The method of claim 4, wherein R¹ is —(CH₂)₄—.

23. The method of claim 1, wherein the compound is selected from the group consisting of (2R,3R,4R,5S)-2-(hydroxymethyl)-1-(9-methoxynonyl)piperidine-3,4,5-triol; (2R,3R,4R,5S)-1-(6-((4-azido-2-nitrophenyl)amino)hexyl)-2-(hydroxymethyl)piperidine-3,4,5-triol; (2R,3R,4R,5S)-2-(hydroxymethyl)-1-(8-(tetrahydrofuran-2-yl)octyl)piperidine-3,4,5-triol; (2R,3R,4R,5S)-2-(hydroxymethyl)-1-(9-methoxy-9-methyldecyl)piperidine-3,4,5-triol; (2R,3R,4R,5S)-2-(hydroxymethyl)-1-(6-((2-nitro-4-(2H-1,2,3-triazol-2-yl)phenyl)amino)hexyl)piperidine-3,4,5-triol; (E)-N′-hydroxy-3-nitro-4-((6-((2R,3R,4R,5S)-3,4,5-trihydroxy-2-(hydroxymethyl)piperidin-1-yl)hexyl)amino)benzimidamide; (2R,3R,4R,5S)-2-(hydroxymethyl)-1-(6-((2-nitro-4-(1H-tetrazol-1-yl)phenyl)amino)hexyl)piperidine-3,4,5-triol; (2R,3R,4R,5S)-2-(hydroxymethyl)-1-(6-((2-nitro-4-(2H-tetrazol-2-yl)phenyl)amino)hexyl)piperidine-3,4,5-triol; (2R,3R,4R,5S)-2-(hydroxymethyl)-1-(6-((2-nitro-4-(pyrimidin-2-yl)phenyl)amino)hexyl)piperidine-3,4,5-triol; (2R,3R,4R,5S)-2-(hydroxymethyl)-1-(6-((4-morpholino-2-nitrophenyl)amino)hexyl)piperidine-3,4,5-triol; (2R,3R,4R,5S)-2-(hydroxymethyl)-1-(6-((2-nitro-5-(1H-tetrazol-1-yl)phenyl)amino)hexyl)piperidine-3,4,5-triol; (2R,3R,4R,5S)-2-(hydroxymethyl)-1-(6-((2-nitro-5-(piperazin-1-yl)phenyl)amino)hexyl)piperidine-3,4,5-triol; (2R,3R,4R,5S)-1-(6-((4-((dimethylamino)methyl)-2-nitrophenyl)amino)hexyl)-2-(hydroxymethyl)piperidine-3,4,5-triol; (2R,3R,4R,5S)-1-(6-((5-azido-2-nitrophenyl)amino)hexyl)-2-(hydroxymethyl)piperidine-3,4,5-triol; (2R,3R,4R,5S)-2-(hydroxymethyl)-1-(6-((3-nitro-4-(1H-pyrazol-1-yl)phenyl)amino)hexyl)piperidine-3,4,5-triol; (2R,3R,4R,5S)-2-(hydroxymethyl)-1-(6-((3-nitro-4-(1H-tetrazol-1-yl)phenyl)amino)hexyl)piperidine-3,4,5-triol; (2R,3R,4R,5S)-2-(hydroxymethyl)-1-(6-((4-((4-methylpiperazin-1-yl)methyl)-2-nitrophenyl)amino)hexyl)piperidine-3,4,5-triol; (2R,3R,4R,5S)-1-(6-((5-((dimethylamino)methyl)-2-nitrophenyl)amino)hexyl)-2-(hydroxymethyl)piperidine-3,4,5-triol; (2R,3R,4R,5S)-1-(4-((4-azido-2-nitrophenyl)amino)butyl)-2-(hydroxymethyl)piperidine-3,4,5-triol; (2R,3R,4R,5S)-2-(hydroxymethyl)-1-(6-((2,4,6-trifluorophenyl)amino)hexyl)piperidine-3,4,5-triol; (2R,3R,4R,5S)-1-butyl-2-(hydroxymethyl)piperidine-3,4,5-triol; (2R,3R,4R,5S)-2-(hydroxymethyl)-1-nonylpiperidine-3,4,5-triol; (2R,3R,4R,5S)-2-(hydroxymethyl)-1-(6-propoxyhexyl)piperidine-3,4,5-triol; (2R,3R,4R,5S)-1-(6-((4-(2H-tetrazol-2-yl)phenyl)amino)hexyl)-2-(hydroxymethyl)piperidine-3,4,5-triol; (2R,3R,4R,5S)-1-(6-((7H-purin-2-yl)amino)hexyl)-2-(hydroxymethyl)piperidine-3,4,5-triol; (2R,3R,4R,5S)-2-(hydroxymethyl)-1-(6-((4-(isoxazolidin-2-ylmethyl)-2-nitrophenyl)amino)hexyl)piperidine-3,4,5-triol; (2R,3R,4R,5S)-2-(hydroxymethyl)-1-(6-((2-nitro-4-(pyridin-2-yl)phenyl)amino)hexyl)piperidine-3,4,5-triol; (2R,3R,4R,5S)-2-(hydroxymethyl)-1-(6-((2-nitro-4-(pyridazin-3-yl)phenyl)amino)hexyl)piperidine-3,4,5-triol; (2R,3R,4R,5S)-2-(hydroxymethyl)-1-(6-((2-nitro-4-(pyrimidin-4-yl)phenyl)amino)hexyl)piperidine-3,4,5-triol; (2R,3R,4R,5S)-2-(hydroxymethyl)-1-(6-((2-nitro-4-(pyridin-3-yl)phenyl)amino)hexyl)piperidine-3,4,5-triol; and pharmaceutically acceptable salts thereof.

24. The method of claim 1, wherein the subject is a human.

25. The method of claim 24, wherein the human is a human female.
